(19) United States
(12) Patent Application Publication
(10) Pub. No.: US 2014/0088152 A1
(43) Pub. Date: Mar. 27, 2014

(54) COMPOSITION FOR PREVENTING AND TREATING CARDIOVASCULAR DISEASES, CONTAINING PYRAZOLE DERIVATIVE

(71) Applicants: Yun Soo Bae, Gyeonggi-do (KR); Kee In Lee, Daejeon (KR); Jee Hyun Lee, Seoul (KR); Han Joong Jo, Seoul (KR); Jinoh Kim, Seoul (KR)

(72) Inventors: Yun Soo Bae, Gyeonggi-do (KR); Kee In Lee, Daejeon (KR); Jee Hyun Lee, Seoul (KR); Han Joong Jo, Seoul (KR); Jinoh Kim, Seoul (KR)

(73) Assignee: EWHA UNIVERSITY-INDUSTRY COLLABORATION FOUNDATION, Seoul (KR)

(21) Appl. No.: 14/002,103

(22) PCT Filed: Feb. 27, 2012
(86) PCT No.: PCT/KR2012/001450

§ 371 Date: Nov. 6, 2013

(30) Foreign Application Priority Data
Feb. 28, 2011 (KR) 1020110017962

(51) Int. Cl. C07D 401/04

(52) U.S. Cl. CPC C07D 401/04
USPC 514/341; 546/274.1

(57) ABSTRACT

The present invention provides a composition comprising a pyrazole derivative compound and a pharmaceutically acceptable salt thereof. The composition is remarkably effective for preventing and treating cardiovascular diseases.

### TECHNICAL FIELD

The present invention relates to a composition for preventing and treating cardiovascular diseases, comprising a pyrazole derivative.

### BACKGROUND ART

Arteriosclerosis, particularly atherosclerosis refers to a condition in which fatty substances (plaques) containing cholesterol, phospholipid, calcium, etc. accumulate on intima, which causes the arteries to harden, lose its elasticity and narrow to inhibit the blood supply or increase the pressure, leading to rupture or dissection of the artery. In particular, arteriostenosis (narrowed blood vessels) occurs due to atherosclerosis, and thus the blood supply is reduced, which results in lack of nutrients and oxygen, thus causing cardiovascular diseases (Libby P, et al., Circulation, 86(6), 47-52, 1992; Lundgren C H, et al., Circulation, 90(4), 1927-1934, 1994; Harker, et al., Ann. NY Acad. Sci., 275, 321-329, 1976). The cardiovascular diseases include heart diseases such as arteriosclerosis, heart failure, hypertensive heart disease, arrhythmia, congenital heart disease, myocardial infarction, angina, etc., vascular diseases such as stroke, peripheral vascular disease, etc., and ischemic cardiovascular diseases in a broad sense.

At present, the treatment of cardiovascular diseases caused by arteriosclerosis may be divided into the treatment for angiogenesis and prevention for vascular stenosis and restenosis by inhibiting the growth of vascular smooth muscle cells.

Percutaneous transluminal coronary angioplasty is to expand a narrowed coronary artery without surgical operation, and its examples include percutaneous coronary balloon angioplasty, percutaneous coronary stent implantation, etc. The percutaneous coronary balloon angioplasty is to improve the blood flow of the coronary artery in the following manner: a guide conduit is inserted through the femur or the arm artery to be located at the entrance of a coronary artery lesion through the aorta; another conduit with a balloon attached to its end is located at the stenosis position of the coronary artery through the inside of the guide conduit; and then the balloon is expanded to compress plaque, thus expanding the narrowed coronary artery. Moreover, the stent implantation is to cover the inner wall of the coronary artery with a wire mesh by locating a wire mesh balloon at the stenosis position and expanding the balloon. The incidence of restenosis after the stent implantation is lower than that of balloon angioplasty. Further, the implanted stent serves as a support for the intima, and thus the stent implantation is widely used for treatment of complications after balloon angioplasty. The interventional procedure using coronary angioplasty is used worldwide since it is more convenient, can further reduce the risk due to general anesthesia, and has a higher success rate than the surgical procedure.

However, restenosis occurs in 10 to 80% of patients treated with coronary angioplasty due to injury of the vascular endothelium, mural thrombosis, migration of vascular smooth muscle cells and fibroblast cells, infiltration of mononuclear cells and lymphocytes, new neointima formation, reendothelialization, apoptosis, etc. Moreover, restenosis frequently occurs in the case of diabetes, old age, recent onset of angina pectoris, unstable angina pectoris, etc. (Leimgruber P P et al., Circulation, vol. 73, 710, 1986).

New percutaneous transluminal coronary angioplasty (PTCA) equipments such as atherectomy, laser angioplasty, high-speed rotational atherectomy (rotablator), cutting balloon angioplasty, irradiation, etc. have been introduced to prevent coronary restenosis. Moreover, various systemic and topical medications such as antiplatelet agents, antithrombotic agents, vasodilators, cytostatic agents, lipid metabolism improving agents, antioxidants, etc. and molecular biological therapies such as gene therapies have been developed and attempted. Among others, systemic medications such as oral administration or intravenous administration are most conveniently used. However, their effects on the prevention of restenosis in animal tests were reported only, but the desired level of the drug in the area that had been subjected to PTCA was not achieved, and the restenosis was not prevented in most clinical trials due to side effects of the drug. Theoretically, the restenosis occurs only at the local coronary artery that has been subjected to PTCA, and thus for the prevention of restenosis, topical medications capable of administering the drug at a high concentration in a site-specific manner is more useful than the systemic medications. Recently, in order to directly administer drugs to the area that is subjected to PTCA, double balloon catheters, dispatch or microporous balloons, etc. have been developed and used in clinic. Moreover, in order to deliver drugs into the area that is subjected to PTCA for a long period of time, trial treatments with slow release microspheres or drug-coated stents have been increasing.

Meanwhile, many studies have recently reported that reactive oxygen species (ROS) generated due to oxidative stress is associated with cardiovascular diseases. Moreover, it is known that stable-state oxygen (triplet oxygen) is converted to highly reactive oxygen species (ROS), such as superoxide radicals, hydroxy radicals, hydrogen peroxide, etc. due to environmental and biochemical factors such as enzyme systems, reductive metabolisms, chemicals, pollutants, photochemical reactions, etc., to oxidize various cellular components such as lipids, proteins, nucleic acids, and DNA, thus causing inflammation or damaging multiple organs (Beckman, J. S. et al., Proc. Natl. Acad, Sci. USA, 87, pp 1620-1624, 1990: Sagar, S. et al., Mol. Cell. Biochem., 111, pp 103-108, 1992: Ames, B. N. et al., Proc. Natl, Acad, USA, 90, pp 7915-7922, 1993). Examples thereof may be superoxide anion (.O₂), hydrogen peroxide (H₂O₂), hydroxyl radicals (.OH), singlet oxygen (¹O₂), alkoxyl radical (RO.) generated during lipid peroxidation, peroxyl radical (ROO.), and nitrogen peroxide ion (ONOO—). It was reported that the action of reactive oxygen is minimized by the action of antioxidative enzymes, such as superoxide dismutase (SOD), catalase, peroxidase, etc. which are biological defense mechanisms, and antioxidants such as vitamins C and E, glutathione, etc., but in the event of a failure in the biological defense mechanism or exposure to an excessive reactive oxygen, the reactive oxygen species irreversibly breaks the lipids, proteins, DNA, etc. due to disruption in balance between the action of reactive oxygen and the action of antioxidative enzymes and antioxidants, thus causing various diseases. In particular, it is known that the reactive oxygen is an important cause of cardiovascular diseases such as multiple atherosclerosis (Griffiths and Lunec, FEES Lett., 388, pp 161-164, 1996: Squadrito, G. L. et al., Free Radic. Biol, Med., 25, pp 392-403, 1998: Choi, J. S. et al., Phytochem. Res., 16, pp 232-235, 2002: Beckman, J. S. et al., Proc. Natl. Acad, Sci, USA., 87, pp 1620-1624, 1990).

Therefore, the inventors of the present invention have made efforts to find a substance useful for the prevention and treatment of cardiovascular diseases and discovered that pyrazole derivatives of the present invention inhibit the growth and migration of smooth muscle cells and further exhibit excellent inhibitory activity on the generation of reactive oxygen species and these compounds can be used for the prevention or treatment of cardiovascular diseases, thereby completing the present invention.

### DISCLOSURE

### Technical Problem

An object of the present invention is to provide a composition for preventing and treating cardiovascular diseases, comprising a pyrazole derivative.

Another object of the present invention is to provide a method for removing vascular stenosis, the method comprising administering the composition to a subject in need thereof.

Still another object of the present invention is to provide a drug delivery system for topical administration of the composition.

### Technical Solution

The present invention provides a pharmaceutical composition for preventing or treating cardiovascular diseases, comprising a compound represented by the following formula 1 or a pharmaceutically acceptable salt thereof:

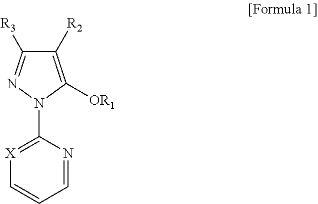

wherein X represents —CH— or nitrogen;

R₁ represents a hydrogen atom or an isopropyloxycarbonyloxymethyl;

R₂ represents a hydrogen atom, a C1-C4 linear or branched alkyl, or a substituted or unsubstituted benzyl; and

R₃ represents a phenyl, a nitrophenyl, a substituted or unsubstituted phenylethenyl, or a substituted or unsubstituted diphenylethenyl;

wherein when R₁ and R₂ each represents a hydrogen atom, R₃ represents a phenyl, a nitrophenyl, or a substituted or unsubstituted diphenylethenyl; and

wherein the substituent is a nitro, a hydroxyl, or a methoxyl.

In the compound represented by formula 1, when X represents —CH— and R₁ and R₂ each represents a hydrogen atom, R₃ may preferably represent a substituted or unsubstituted diphenylethenyl, or when X represents a nitrogen atom and R₁ and R₂ each represents a hydrogen atom, R₃ may preferably represent a nitrophenyl or a substituted or unsubstituted diphenylethenyl. In the salt of the compound represent by formula 1, when R₁ and R₂ each represents a hydrogen atom, R₃ may preferably represent a phenyl, a nitrophenyl, or a substituted or unsubstituted diphenylethenyl.

The compound represented by formula 1 may preferably be selected from:
1-(pyridin-2-yl)-3-phenyl-4-propyl-1H-pyrazol-5-ol (211);
1-(pyridin-2-yl)-3-phenyl-4-benzyl-1H-pyrazol-5-ol (212);
1-(pyridin-2-yl)-3-phenyl-4-(4-nitrobenzyl)-1H-pyrazol-5-ol (225);
1-(pyridin-2-yl)-3-phenyl-4-propyl-1H-pyrazol-5-ol hydrochloride (278);
1-(pyridin-2-yl)-3-phenyl-4-propyl-1H-5-(isopropyloxycarbonyloxymethyloxy)pyrazole (306);
1-(pyridin-2-yl)-3-[(3-methoxy-4-hydroxyphenyl)-E-ethenyl]-4-benzyl-1H-pyrazol-5-ol hydrochloride (308);
1-(pyridin-2-yl)-3-[(3-methoxy-4-hydroxyphenyl)-E-ethenyl]-4-benzyl-5-hydroxypyrazole (274);
3-(4-nitrophenyl)-3-[2-(pyrimidin-2-yl)hydrazono]propionic acid ethyl ester;
1-(pyrimidin-2-yl)-3-(4-nitrophenyl)-5-1H-pyrazol-5-ol (105);
1-(pyridin-2-yl)-3-(1,2-diphenyl-E-ethenyl)-1H-pyrazol-5-ol (263);
1-(pyridin-2-yl)-3-phenyl-1H-pyrazol-5-ol hydrochloride (305);
3-(4-methoxyphenylamino)-1H-pyrazol-5-ol (15); or
4-hexylidene-3-phenyl-1-(pyridin-2-yl)-1H-pyrazol-5-ol (213).

Meanwhile, the compound represented by formula 1 or a pharmaceutically acceptable salt thereof can be prepared by a preparation method comprising:

adding dropwise a compound represented by the following formula 2 and a compound represented by the following formula 3 to a polar organic solvent; and

heating the polar organic solvent comprising the compound represented by formula 2 and the compound represented by formula 3:

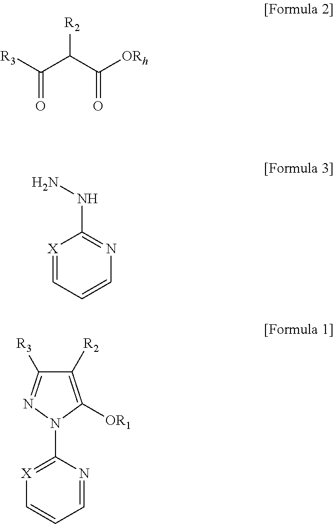

wherein X represents —CH— or nitrogen;

Rₕ represents a C1-C4 linear or branched alkyl group;

R₁ represents a hydrogen atom or an isopropyloxycarbonyloxymethyl;

R₂ represents a hydrogen atom, a C1-C4 linear or branched alkyl, or a substituted or unsubstituted benzyl; and

R₃ represents a phenyl, a nitrophenyl, a substituted or unsubstituted phenylethenyl, or a substituted or unsubstituted diphenylethenyl;

wherein when R₁ and R₂ each represents a hydrogen atom, R₃ represents a phenyl, a nitrophenyl, or a substituted or unsubstituted diphenylethenyl; and

wherein the substituent is a nitro, a hydroxyl, or a methoxyl.

In the preparation of the compound of formula 1, α-substituted β-keto ester, which is the compound represented by formula 2 used as a starting material, is commercially available or may be prepared according to the method described in J. Org. Chem., Vol. 43, No. 10, 1978, 2087-2088, specifically by reacting a commercially available acyl chloride derivative with Meldrum's acid and heating the resulting product under reflux in the presence of an organic solvent such as methanol or ethanol to form β-keto ester. The α-substituted β-keto ester may be prepared according to the method described in J. Chem. Soc., Perkin Trans. 1, 1986, 1139-1143. More specifically, it can be easily prepared by reaction of β-keto ester with alkyl halide in the presence of potassium carbonate or cesium carbonate.

The compound represented by formula 3 as a reactant may be commercially available and may be used in an amount of about 1 to 3 molar equivalents, preferably about 1 to 1.3 molar equivalents, based on 1 molar equivalent of the compound represented by formula 2 as a starting material.

The polar organic solvent may be C1-C4 alcohol such as methanol, ethanol, n-propanol, i-isopropanol, n-butanol or t-butanol, acetic acid, or a mixture thereof and may preferably be ethanol or acetic acid.

The salt of the compound represented by formula 1 may be prepared by reacting the compound represented by formula 1 with an acid material. The acid material is not particularly limited as long as it can form a salt by reaction with the compound represented by formula 1. For example, the acid material may be inorganic acid such as hydrochloric acid, nitric acid, phosphoric acid, bromic acid, iodic acid, perchloric acid, tartaric acid, and sulfuric acid; organic acid such as acetic acid, trifluoroacetic acid, citric acid, maleic acid, succinic acid, oxalic acid, benzoic acid, tartaric acid, fumaric acid, mandelic acid, propionic acid, citric acid, lactic acid, glycolic acid, gluconic acid, galacturonic acid, glutamic acid, glutaric acid, glucuronic acid, aspartic acid, ascorbic acid, carbonic acid, vanillic acid, and hydroiodic acid; or sulfonic acid such as methanesulfonic acid, ethanesulfonic acid, benzenesulfonic acid, p-toluenesulfonic acid, and naphthalene sulfonic acid and may preferably be hydrochloric acid.

The compound represented by formula 2 and the compound represented by formula 3 may be added dropwise to a polar organic solvent at −4° C. to 10° C.

The organic solvent comprising the compound represented by formula 2 and the compound represented by formula 3 may be heated at a reflux temperature of the solvent, preferably at a temperature of about 100 to about 130° C.

The organic solvent comprising the compound represented by formula 2 and the compound represented by formula 3 may be heated for about 10 minutes to 72 hours.

A compound represented by the following formula 4 may be prepared by reaction of the compound represented by formula 2 and with compound represented by formula 3 in the presence of an organic solvent:

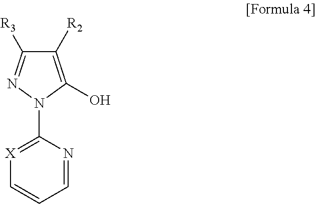

wherein R₂ represents a hydrogen atom, a C1-C4 linear or branched alkyl, or a substituted or unsubstituted benzyl; and

R₃ represents a phenyl, a nitrophenyl, a substituted or unsubstituted phenylethenyl, or a substituted or unsubstituted diphenylethenyl;

wherein the substituent is a nitro, a hydroxyl, or a methoxyl; and

wherein when R₂ represents a hydrogen atom, R₃ represents a phenyl, a nitrophenyl, or a substituted or unsubstituted diphenylethenyl.

The compound represented by formula 4 may react with a halide compound such as isopropyloxycarbonyloxymethyliodide, isopropyloxycarbonyloxymethylchloride, or isopropyloxycarbonyloxymethylbromide in the presence of a base to prepare the compound represented by the formula 1 comprising an isopropyloxycarbonyloxymethyloxy group bonded to the carbon at the 5 position of pyrazole.

In the reaction of the compound represented by formula 4 with the halide compound, the base may be 4-dimethylaminopyridine (DMAP), pyridine, triethylamine, imidazole, a metal salt of carbonate such as carbonate potassium, carbonate sodium, or carbonate calcium, or a mixture thereof. The base may be used in an amount of about 2 to 3 molar equivalents based on 1 molar equivalent of the compound represented by formula 2.

In the reaction of the compound represented by formula 4 with the halide compound, the reaction solvent may be a mixture of water and an organic solvent, preferably a mixture of water and at least one organic solvent selected from methylene chloride, ethyl ether, ethyl acetate, tetrahydrofuran (THF), and N,N′-dimethylformamide (DMF), more preferably a mixture of water and methylene chloride.

The reaction of the compound represented by formula 4 with the halide compound may be carried out in the presence of a phase transfer catalyst. In this case, the compound represented by formula 1 can be obtained in higher purity by preventing the generation of impurities comprising an alkylated amine group at the 2 position of the pyrazole group.

In the reaction of the compound represented by formula 4 with the halide compound, the reaction temperature may preferably be about 0 to about 40° C., more preferably 15 to 30° C., and the reaction time may preferably be 10 to 12 hours. However, depending on the reaction rate, the reaction temperature may be further increased, and the reaction time may be further increased.

The present invention provides a pharmaceutical composition for preventing or treating cardiovascular diseases, comprising a compound represented by the following formula I or a pharmaceutically acceptable salt thereof:

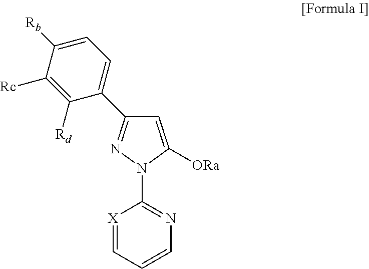

wherein X represents —CH— or nitrogen;

Rₐ represents a hydrogen atom, an acetyl group, a tri(C1-C4)alkylsilanyl group, a diphenylboranyl group, or a (t-butoxy)carbonyl group; and

R_{b}, R_{c} and R_{d} each independently represents a hydrogen atom, a halogen atom (F, Cl, Br, or I), a halo(C1-C3)alkyl group, a (C2-C6)alkoxy group, a benzo[d][1,3]dioxol group, a substituted or unsubstituted biphenyl group, or a substituted or unsubstituted (C6-C10)aryl group, and

wherein the substituent is a halogen atom, a (C1-C4)alkyl amine group, a halo(C1-C3)alkyl group, a (C1-C6)alkoxy group, a phenoxy group, a benzyloxy group, a formyl group, or a halogen-substituted phenyl group, with the proviso that R_{b}, R_{C} and R_{d} are not a hydrogen atom at the same time.

The compound represented by formula I may be selected from:
1-(pyridin-2-yl)-3-(3-(trifluoromethyl)phenyl)-1H-pyrazol-5-ol;
1-(pyridin-2-yl)-3-(4-(trifluoromethyl)phenyl)-1H-pyrazol-5-ol;
3-(3-nitrophenyl)-1-(pyridin-2-yl)-1H-pyrazol-5-ol;
1-(pyridin-2-yl)-3-(3,4,5-trimethoxyphenyl)-1H-pyrazol-5-ol;
3-(2-fluorophenyl)-1-(pyridin-2-yl)-1H-pyrazol-5-ol;
3-(3-fluorophenyl)-1-(pyridin-2-yl)-1H-pyrazol-5-ol;
3-(4-fluorophenyl)-1-(pyridin-2-yl)-1H-pyrazol-5-ol;
3-(2-chlorophenyl)-1-(pyridin-2-yl)-1H-pyrazol-5-ol;
3-(3-chlorophenyl)-1-(pyridin-2-yl)-1H-pyrazol-5-ol;
3-(4-chlorophenyl)-1-(pyridin-2-yl)-1H-pyrazol-5-ol;
3-(3-bromophenyl)-1-(pyridin-2-yl)-1H-pyrazol-5-ol;
3-(4-bromophenyl)-1-(pyridin-2-yl)-1H-pyrazol-5-ol;
3-(2-iodophenyl)-1-(pyridin-2-yl)-1H-pyrazol-5-ol;
3-(3-iodophenyl)-1-(pyridin-2-yl)-1H-pyrazol-5-ol;
3-(4-iodophenyl)-1-(pyridin-2-yl)-1H-pyrazol-5-ol;
3-(4-iodophenyl)-1-(pyrimidin-2-yl)-1H-pyrazol-5-ol;
2-(3-(4-iodophenyl)-5-(triisopropylsilyloxy)-1H-pyrazol-1-yl)pyridine;
3-(4-bromophenyl)-1-(pyridin-2-yl)-1H-pyrazol-5-yl t-butyl carbonate;
t-butyl 3-(2-iodophenyl)-1-(pyridin-2-yl)-1H-pyrazol-5-yl carbonate;
t-butyl 3-(3-iodophenyl)-1-(pyridin-2-yl)-1H-pyrazol-5-yl carbonate;
t-butyl 3-(4-iodophenyl)-1-(pyridin-2-yl)-1H-pyrazol-5-yl carbonate;
t-butyl 3-(4-iodophenyl)-1-(pyrimidin-2-yl)-1H-pyrazol-5-yl carbonate;
3-(4-bromophenyl)-1-(pyridin-2-yl)-1H-pyrazol-5-yl acetate;
3-(biphenyl-4-yl)-1-(pyridin-2-yl)-1H-pyrazol-5-yl t-butyl carbonate;
3-(4-(benzo[d][1,3]dioxol-5-yl)phenyl)-1-(pyridin-2-yl)-1H-pyrazol-5-yl t-butyl carbonate;
3-(3′-phenylbiphenyl-4-yl)-1-(pyridin-2-yl)-1H-pyrazol-5-yl t-butyl carbonate;
t-butyl 3-(4-(naphthalene)phenyl)-1-(pyridin-2-yl)-1H-pyrazol-5-yl carbonate;
t-butyl 3-(3′-(dimethylamino)biphenyl)-1-(pyridin-2-yl)-1H-pyrazol-5-yl carbonate;
3-(2-(benzo[d][1,3]dioxol-5-yl)phenyl)-1-(pyridin-2-yl)-1H-pyrazol-5-yl t-butyl carbonate;
3-(3′-phenylbiphenyl-2-yl)-1-(pyridin-2-yl)-1H-pyrazol-5-yl t-butyl carbonate;
3-(biphenyl-3-yl)-1-(pyridin-2-yl)-1H-pyrazol-5-yl t-butyl carbonate;
3-(4′-(benzyloxy)biphenyl-3-yl)-1-(pyridin-2-yl)-1H-pyrazol-5-yl t-butyl carbonate;
3-(4′-bromobiphenyl-3-yl)-1-(pyridin-2-yl)-1H-pyrazol-5-yl t-butyl carbonate;
t-butyl 3-(3′-formylbiphenyl-3-yl)-1-(pyridin-2-yl)-1H-pyrazol-5-yl carbonate;
t-butyl 3-(2′-phenoxybiphenyl-3-yl)-1-(pyridin-2-yl)-1H-pyrazol-5-yl carbonate;
3-(3-(benzo[d][1,3]dioxol-5-yl)phenyl)-1-(pyridin-2-yl)-1H-pyrazol-5-yl t-butyl carbonate;
3-(3′-phenylbiphenyl-3-yl)-1-(pyridin-2-yl)-1H-pyrazol-5-yl t-butyl carbonate;
t-butyl 3-(3-(naphthalene-1-yl)phenyl)-1-(pyridin-2-yl)-1H-pyrazol-5-yl carbonate;
t-butyl 3-(3′-(dimethylamino)biphenyl-3-yl)-1-(pyridin-2-yl)-1H-pyrazol-5-yl carbonate;
t-butyl 3-(4′-methoxybiphenyl-4-yl)-1-(pyridin-2-yl)-1H-pyrazol-5-yl carbonate;
3-(4′-(benzyloxy)biphenyl-4-yl)-1-(pyridin-2-yl)-1H-pyrazol-5-yl t-butyl carbonate;
3-(4′-bromobiphenyl-4-yl)-1-(pyridin-2-yl)-1H-pyrazol-5-yl t-butyl carbonate;
3-(3′-formylbiphenyl-4-yl)-1-(pyridin-2-yl)-1H-pyrazol-5-yl t-butyl carbonate;
t-butyl 3-(2′-phenoxybiphenyl-4-yl)-1-(pyridin-2-yl)-1H-pyrazol-5-yl carbonate;
3-(biphenyl-4-yl)-1-(pyrimidin-2-yl)-1H-pyrazol-5-yl t-butyl carbonate;
3-(4-(benzo[d][1,3]dioxol-5-yl)phenyl)-1-(pyrimidin-2-yl)-1H-pyrazol-5-yl t-butyl carbonate;
3-(3′-phenylbiphenyl-4-yl)-1-(pyrimidin-2-yl)-1H-pyrazol-5-yl t-butyl carbonate;
2-(3-(4′-bromobiphenyl-4-yl)-5-(triisopropylsilyloxy)-1H-pyrazol-1-yl)pyridine;
2-(3-(biphenyl-4-yl)-5-(diphenylboryloxy)-1H-pyrazol-1-yl)pyridine;
3-(biphenyl-4-yl)-1-(pyridin-2-yl)-1H-pyrazol-5-ol;
3-(4-(benzo[d][1,3]dioxol-5-yl)phenyl)-1-(pyridin-2-yl)-1H-pyrazol-5-ol;
3-(3′-phenylbiphenyl-4-yl)-1-(pyridin-2-yl)-1H-pyrazol-5-ol;
3-(4-(naphthalene-1-yl)phenyl)-1-(pyridin-2-yl)-1H-pyrazol-5-ol;
3-(3′-(dimethylamino)biphenyl-4-yl)-1-(pyridin-2-yl)-1H-pyrazol-5-ol;
3-(2-(benzo[d][1,3]dioxol-5-yl)phenyl)-1-(pyridin-2-yl)-1H-pyrazol-5-ol;
3-(3′-phenylbiphenyl-2-yl)-1-(pyridin-2-yl)-1H-pyrazol-5-ol;
3-(biphenyl-3-yl)-1-(pyridin-2-yl)-1H-pyrazol-5-ol;
3-(4′-(benzyloxy)biphenyl-3-yl)-1-(pyridin-2-yl)-1H-pyrazol-5-ol;
3-(4′-bromobiphenyl-3-yl)-1-(pyridin-2-yl)-1H-pyrazol-5-ol;
3-(3′-formylbiphenyl-3-yl)-1-(pyridin-2-yl)-1H-pyrazol-5-ol;
3-(2′-phenoxybiphenyl-3-yl)-1-(pyridin-2-yl)-1H-pyrazol-5-ol;
3-(3-(benzo[d][1,3]dioxol-5-yl)phenyl)-1-(pyridin-2-yl)-1H-pyrazol-5-ol;
3-(3′-phenylbiphenyl-3-yl)-1-(pyridin-2-yl)-1H-pyrazol-5-ol;
3-(3-(naphthalene-1-yl)phenyl)-1-(pyridin-2-yl)-1H-pyrazol-5-ol;
3-(3′-(dimethylamino)biphenyl-3-yl)-1-(pyridin-2-yl)-1H-pyrazol-5-ol;
3-(4′-methoxybiphenyl-4-yl)-1-(pyridin-2-yl)-1H-pyrazol-5-ol;
3-(4′-(benzyloxy)biphenyl-4-yl)-1-(pyridin-2-yl)-1H-pyrazol-5-ol;
3-(4′-bromobiphenyl-4-yl)-1-(pyridin-2-yl)-1H-pyrazol-5-ol;
3-(3′-formylbiphenyl-4-yl)-1-(pyridin-2-yl)-1H-pyrazol-5-ol;
3-(2′-phenoxybiphenyl-4-yl)-1-(pyridin-2-yl)-1H-pyrazol-5-ol;
3-(biphenyl-4-yl)-1-(pyrimidin-2-yl)-1H-pyrazol-5-ol;
3-(4-(benzo[d][1,3]dioxol-5-yl)phenyl)-1-(pyrimidin-2-yl)-1H-pyrazol-5-ol;
3-(3′-phenylbiphenyl-4-yl)-1-(pyridin-2-yl)-1H-pyrazol-5-ol;
3-phenyl-4-propyl-1-(pyridin-2-yl)-1H-pyrazol-5-ol; and
4-benzyl-3-phenyl-1-(pyridin-2-yl)-1H-pyrazol-5-ol.

Meanwhile, the compound represented by formula I or a pharmaceutically acceptable salt thereof may be prepared by the following method.

Specifically, a compound represented by the following formula I-1 corresponding to formula I or a pharmaceutically acceptable salt thereof may be prepared by a method including adding a compound represented by the following formula II and a compound represented by the following formula III to a polar organic solvent and heating the resulting polar organic solvent to prepare the compound of formula I-1:

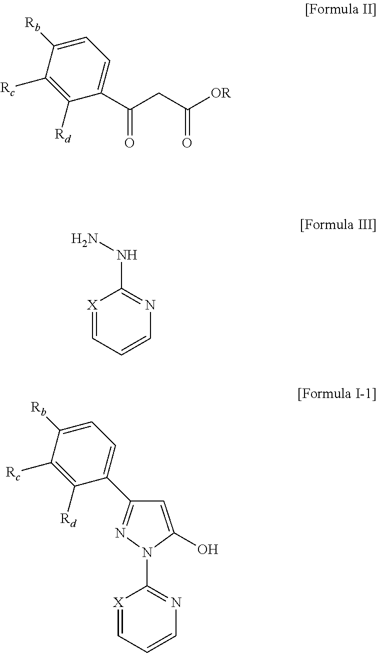

wherein X represents —CH— or nitrogen;

R represents a (C1-C4)alkyl group; and

R_{b}, R_{c} and R_{d} each independently represents a hydrogen atom, a halogen atom, a halo(C1-C3)alkyl group, or a (C2-C6)alkoxy group, with the proviso that R_{b}, R_{c} and R_{d} are not a hydrogen atom at the same time.

In the method for preparing the compound of formula I-1, β-keto ester, which is the compound of formula II used as a starting material, may be commercially available or may be prepared according to the method described in J. Org. Chem., Vol. 43, No, 10, 1978, 2087-2088, specifically by reacting a commercially available acyl chloride derivative with Meldrum's acid and heating the resulting product under reflux in the presence of an organic solvent such as methanol or ethanol.

In the method for preparing the compound of formula I-1, the compound of formula III as a reactant may be commercially available and may be used in an amount of about 1 to 3 molar equivalents, preferably about 1 to 1.3 molar equivalents, based on 1 molar equivalent of the compound of formula II as a starting material.

In the method for preparing the compound of formula I-1, the polar organic solvent may be preferably selected from C1-C4 alcohol such as methanol, ethanol, n-propanol, isopropanol, n-butanol or t-butanol, acetic acid, and a mixture thereof. Ethanol or acetic acid is more preferred.

In the method for preparing the compound of formula I-1, the heating may preferably be carried out at a reflux temperature of the solvent, preferably at a temperature of about 100 to about 130° C., for example.

In the method for preparing of the compound of formula I-1, the reaction may preferably be carried out for 2 to 72 hours.

The compound represented by the following formula I-2 corresponding to formula I or a pharmaceutically acceptable salt thereof may be prepared by a method including reacting the compound of formula I-1 with one selected from acetyl chloride, tris(C1-C4)alkylsilyl chloride, or di-t-butyl dicarbonate (BOC₂O) in the presence of a base to prepare the compound of formula I-2:

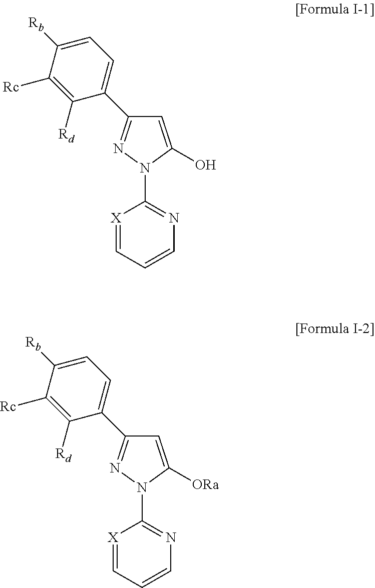

wherein X represents —CH— or nitrogen;

Rₐ represents an acetyl group, a tri(C1-C4)alkylsilyl group, or a (t-butoxy)carbonyl group; and

R_{b}, R_{c} and R_{d} each independently represents a hydrogen atom, a halogen atom, a halo(C1-C3)alkyl group, or a (C2-C6)alkoxy group, with the proviso that R_{b}, R_{c} and R_{d} are not a hydrogen atom at the same time.

In the method for preparing the compound of formula I-2, acetyl chloride, tris(C1-C4)alkylsilyl chloride, or di-t-butyl dicarbonate (BOC₂O) as a reactant may preferably be used in an amount of 1.2 to 5 equivalents based on 1 equivalent of the compound of formula I-1 as a starting material.

In the method for preparing the compound of formula I-2, the base may preferably be selected from 4-dimethylaminopyridine (DMAP), pyridine, triethylamine, and imidazole, and the catalyst may more preferably be 4-dimethylaminopyridine. Here, the base may preferably be used in an amount of 2 to 3 equivalents, and the catalyst may preferably be used in an amount of 0.01 to 0.5 molar equivalents, more preferably 0.05 molar equivalents, based on 1 molar equivalent of the compound of formula I-1 as a starting material.

In the method for preparing the compound of formula I-2, the reaction solvent may preferably be, for example, an organic solvent such as methylene chloride, ethyl ether, ethyl acetate, tetrahydrofuran (THF), or N,N′-dimethylformamide (DMF), more preferably methylene chloride.

In the method for preparing the compound of formula I-2, the reaction temperature may preferably be about 0 to about 40° C., more preferably 15 to 30° C., and the reaction time may preferably be 10 to 12 hours. However, depending on the reaction rate, the reaction temperature may be further increased, and the reaction time may be further increased.

Moreover, a compound represented by the following formula I-3 corresponding to formula I or a pharmaceutically acceptable salt thereof may be prepared by a method including reacting the compound of formula I-2 with a compound of formula IV in the presence of a palladium metal catalyst and a base to prepare the compound of formula I-3:

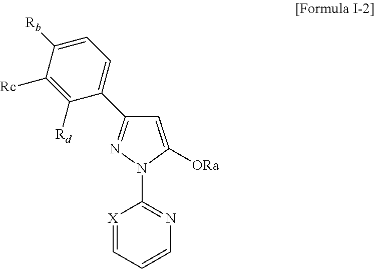

wherein in formula I-2, X represents —CH— or nitrogen;

Rₐ represents a hydrogen, an acetyl group, a tri(C1-C4)alkylsilanyl group, or a (t-butoxy)carbonyl group; and

R_{b}, R_{c} and R_{d} each independently represents a hydrogen atom or a halogen atom, with the proviso that R_{b}, R_{c} and R_{d} are not a hydrogen atom at the same time,

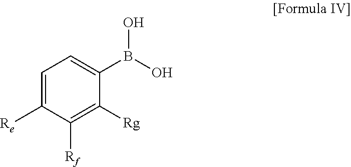

wherein in formula IV, Rₑ, R_{f} and R_{g} each independently represents a hydrogen atom, a halogen atom, a (C1-C4) alkylamine group, a halo(C1-C3)alkyl group, a (C1-C6)alkoxy group, a phenoxy group, a benzyloxy group, a formyl group, a phenyl group, or a halogen-substituted phenyl group, or alternatively Rₑ and R_{f} or R_{f} and R_{g} represent —OCH₂O— or —CH═CH—CH═CH—, with the proviso that Rₑ, R_{f} and R_{g} are not a hydrogen atom at the same time, and

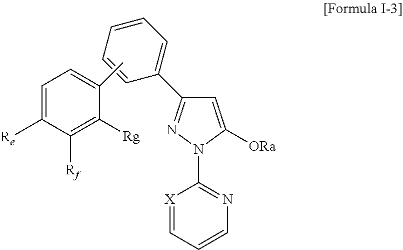

wherein in formula I-3, X represents —CH— or nitrogen;

Rₐ represents a hydrogen, an acetyl group, a tri(C1-C4)alkylsilanyl group, a diphenylboranyl group, or a (t-butoxy)carbonyl group; and

Rₑ, R_{f} and R_{g} each independently represents a hydrogen atom, a halogen atom, a (C1-C4)alkylamine group, a halo(C1-C3)alkyl group, a (C1-C6)alkoxy group, a phenoxy group, a benzyloxy group, a formyl group, a phenyl group, or a halogen-substituted phenyl group, or alternatively Rₑ and R_{f} or R_{f} and R_{g} represent —OCH₂O— or —CH═CH—CH═CH—, with the proviso that Rₑ, R_{f} and R_{g} are not a hydrogen atom at the same time.

In the method for preparing the compound of formula I-3, the compound of formula IV as a reactant may be commercially available. The compound of formula IV as a reactant may preferably be used in an amount of about 1 to 5 molar equivalents, more preferably 2 to 3 molar equivalents, based on 1 molar equivalent of the compound of formula I-2.

In the method for preparing the compound of formula I-3, the palladium metal catalyst may preferably be PdCl₂(dppf) and 1,1′-bis(diphenylphosphino)ferrocene (dppf), or an available Pd catalyst may preferably be Pd(PPh₃)₄, Pd(OAc)₂, Pd(dba)CHCl₃, etc., more preferably PdCl₂(dppf) and 1,1′-bis(diphenylphosphino)ferrocene (dppf). Here, the palladium metal catalyst may preferably be used in an amount of 0.01 to 0.5 equivalents, more preferably 0.03 to 0.1 equivalents, based on 1 equivalent of the compound of formula I-2. Moreover, PdCl₂(dppf) and 1,1′-bis(diphenylphosphino)ferrocene (dppf) may preferably be used in an equivalent ratio of 2:1.

In the method for preparing the compound of formula I-3, the base may preferably be selected from K₃PO₄, K₂CO₃, Ba(OH)₂, and Cs₂CO₃ and may preferably be used in an amount of 1 to 3 molar equivalents, based on 1 molar equivalent of the compound of formula I-2.

In the method for preparing the compound of formula I-3, the reaction temperature may preferably be 90 to 110° C., and the reaction solvent may preferably be selected from 1,4-dioxane, THF, DMF, and toluene.

Moreover, a compound represented by the following formula I-4 corresponding to formula I or a pharmaceutically acceptable salt thereof may be prepared by a method including converting the compound of formula I-3 into a compound of formula I-4 in the presence of an organic acid:

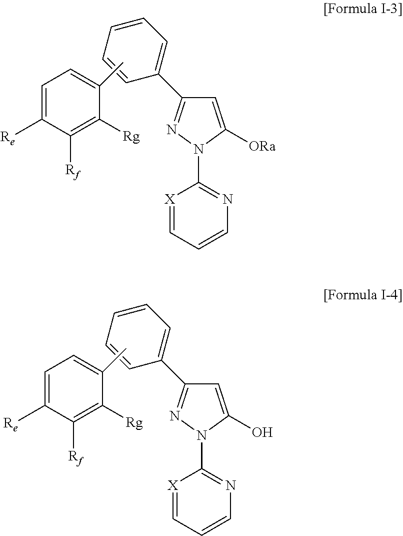

wherein X represents —CH— or nitrogen;

Rₐ represents an acetyl group, a tri(C1-C4)alkylsilanyl group, or a (t-butoxy)carbonyl group; and

Rₑ, R_{f} and R_{g} each independently represents a hydrogen atom, a halogen atom, a (C1-C4)alkylamine group, a halo(C1-C3)alkyl group, a (C1-C6)alkoxy group, a phenoxy group, a benzyloxy group, a formyl group, a phenyl group, or a halogen-substituted phenyl group, or alternatively Rₑ and R_{f} or R_{f} and R_{g} represent —OCH₂O— or —CH═CH—CH═CH—, with the proviso that Rₑ, R_{f} and R_{g} are not a hydrogen atom at the same time.

In the method for preparing the compound of formula I-4, the organic acid may preferably be selected from trifluoroacetic acid, trichloroacetic acid, HF, and HCl, and trifluoroacetic acid is more preferred. Here, the organic acid may preferably be used in an amount of about 2 to 10 equivalents, more preferably 4 to 6 equivalents, based on 1 equivalent of the compound of formula I-3.

In the method for preparing the compound of formula I-4, the reaction temperature may preferably be about 0 to 40° C., more preferably 15 to 30° C. The reaction solvent may preferably be selected from methylene chloride, THF, chloroform, and dichloroethane.

The present invention provides a pharmaceutical composition for preventing or treating cardiovascular diseases, containing a compound represented by the following formula A or a pharmaceutically acceptable salt thereof:

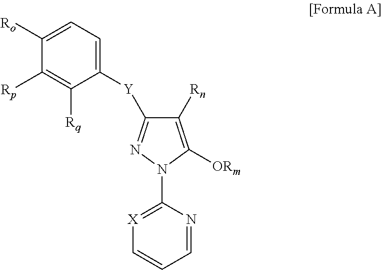

wherein X represents —CH— or nitrogen;

Y represents —CH₂—, —CH₂CH₂—, —CH═CH—, —CH₂—O—, or —O—CH₂—;

Rₘ represents a hydrogen atom, an acetyl group, a tri(C1-C4)alkylsilanyl group, a diarylboranyl group, or a (t-butoxy)carbamyl group;

Rₙ represents a hydrogen atom or a (C1-C4)alkyl group; and

Rₒ, Rₚ and R_{q} each independently represents a hydrogen atom, a hydroxyl group, a halogen atom, a (C6-C10)aryl group, a halo(C1-C3)alkyl group, a (C1-C6)alkoxy group, a tri(C1-C4)alkylsilanoxy group, or a benzodioxolyl group, or alternatively R₁ and Rₚ or Rₚ and R_{q} together represent —CH₂—CH═CH—, —CH═CH—CH═CH—, or —CH═CH—CH═CH—CH₂—.

In the compound represent by formula A, X may represent —CH— or nitrogen, Y may represent —CH₂—, —CH₂CH₂—, —CH═CH—, —CH₂—O—, or —O—CH₂—, Rₘ and Rₙ each may represent a hydrogen atom, and Rₒ, Rₚ and R_{q} each may independently represent a hydrogen atom, a hydroxyl group, a halogen atom, a (C6-C10)aryl group, a halo(C1-C3)alkyl group, a (C1-C6)alkoxy group, a tri(C1-C4)alkylsilanoxy group, or a benzodioxolyl group, or alternatively Rₙ and Rₚ or Rₚ and R_{q} together may represent —CH₂—CH═CH—, —CH═CH—CH═CH—, or —CH═CH—CH═CH—CH₂—.

In the compound represent by formula A, when X represents —CH═CH—, it may preferably be a trans or cis isomer, more preferably a trans isomer.

The compound represented by formula A may be selected from:
3-benzyl-1-(pyridin-2-yl)-1H-pyrazol-5-ol;
3-(o-bromobenzyl)-1-(pyridin-2-yl)-1H-pyrazol-5-ol;
3-(p-methoxybenzyl)-1-(pyridin-2-yl)-1H-pyrazol-5-ol;
3-phenethyl-1-(pyridin-2-yl)-1H-pyrazol-5-ol;
3-(p-chlorobenzyl)-1-(pyridin-2-yl)-1H-pyrazol-5-ol;
3-(phenoxymethyl)-1-(pyridin-2-yl)-1H-pyrazol-5-ol;
3-(naphthalene-3-yloxy)methyl)-1-(pyridin-2-yl)-1H-pyrazol-5-ol;
3-((4-chlorophenoxy)methyl)-1-(pyridin-2-yl)-1H-pyrazol-5-ol;
3-((2,4-dichlorophenoxy)methyl)-1-(pyridin-2-yl)-1H-pyrazol-5-ol;
1-(pyridin-2-yl)-3-styryl-1H-pyrazol-5-ol;
3-(4-methoxystyryl)-1-(pyridin-2-yl)-1H-pyrazol-5-ol;
3-(3,4-dimethoxystyryl)-1-(pyridin-2-yl)-1H-pyrazol-5-ol;
3-(3,4-dichlorostyryl)-1-(pyridin-2-yl)-1H-pyrazol-5-ol;
3-(4-i-propylstyryl)-1-(pyridin-2-yl)-1H-pyrazol-5-ol;
3-(4-trifluoromethylstyryl)-1-(pyridin-2-yl)-1H-pyrazol-5-ol;
3-(4-t-butyldimethylsilyloxystyryl)-1-(pyridin-2-yl)-1H-pyrazol-5-ol;
3-(3-methoxy-4-t-butyldimethylsilyloxystyryl)-1-(pyridin-2-yl)-1H-pyrazol-5-ol;
3-(3,5-dimethoxy-4-t-butyldimethylsilyloxystyryl)-1-(pyridin-2-yl)-1H-pyrazol-5-ol;
3-(3-methoxy-4-t-butyldimethylsilyloxystyryl)-1-(pyridin-2-yl)-1H-pyrazol-5-thiol;
3-(4-hydroxystyryl)-1-(pyridin-2-yl)-1H-pyrazol-5-ol;
3-(3-methoxy-4-hydroxystyryl)-1-(pyridin-2-yl)-1H-pyrazol-5-ol;
3-(3,5-dimethoxy-4-hydroxystyryl)-1-(pyridin-2-yl)-1H-pyrazol-5-ol;
3-(3-methoxy-4-hydroxystyryl)-1-(pyridin-2-yl)-1H-pyrazol-5-thiol; and
4-((E)-2-(5-amino-1-(pyridin-2-yl)-1H-pyrazol-3-yl)vinyl)-2-methoxyphenol.

The compound represented by formula A may more preferably be a compound selected from:
3-phenethyl-1-(pyridin-2-yl)-1H-pyrazol-5-ol,
3-(p-chlorobenzyl)-1-(pyridin-2-yl)-1H-pyrazol-5-ol,
3-(phenoxymethyl)-1-(pyridin-2-yl)-1H-pyrazol-5-ol,
3-(naphthalene-3-yloxy)methyl)-1-(pyridin-2-yl)-1H-pyrazol-5-ol,
3-((2,4-dichlorophenoxymethyl)-1-(pyridin-2-yl)-1H-pyrazol-5-ol,
1-(pyridin-2-yl)-3-styryl-1H-pyrazol-5-ol,
3-(4-methoxystyryl)-1-(pyridin-2-yl)-1H-pyrazol-5-ol,
3-(3,4-dimethoxystyryl)-1-(pyridin-2-yl)-1H-pyrazol-5-ol,
3-(3,4-dichlorostyryl)-1-(pyridin-2-yl)-1H-pyrazol-5-ol,
3-(4-trifluoromethylstyryl)-1-(pyridin-2-yl)-1H-pyrazol-5-ol,
3-(4-t-butyldimethylsilyloxystyryl)-1-(pyridin-2-yl)-1H-pyrazol-5-ol,
3-(4-hydroxystyryl)-1-(pyridin-2-yl)-1H-pyrazol-5-ol; and
3-(3-methoxy-4-hydroxystyryl)-1-(pyridin-2-yl)-1H-pyrazol-5-thiol;

or a pharmaceutically acceptable salt thereof.

Meanwhile, the compound represented by formula A or a pharmaceutically acceptable salt thereof may be prepared by a method including adding a compound represented by the following formula B and 2-hydrazinopyridine to a polar organic solvent and heating the resulting polar organic solvent to prepare the compound of formula A:

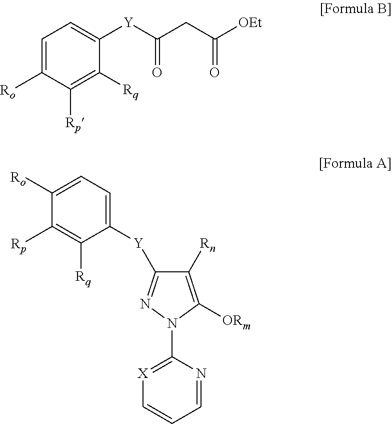

wherein X represents —CH—;

Y represents —CH₂—, —CH₂CH₂—, —CH═CH—, —CH₂—O—, or —O—CH₂—;

Rₘ represents a hydrogen atom, an acetyl group, a tri(C1-C4)alkylsilanyl group, a diarylboranyl group, or a (t-butoxy)carbamyl group;

Rₙ represents a hydrogen atom or a (C1-C4)alkyl group; and

Rₒ, Rₚ and R_{q} each independently represents a hydrogen atom, a hydroxyl group, a halogen atom, a (C6-C10)aryl group, a halo(C1-C3)alkyl group, a (C1-C6)alkoxy group, a tri(C1-C4)alkylsilanoxy group, or a benzodioxolyl group, or alternatively Rₒ and Rₚ or Rₚ and R_{q} together represent —CH₂—CH═CH—, —CH═CH—CH═CH—, or —CH═CH—CH═CH—CH₂—.

It is more preferable to prepare a compound of formula A in which X represents —CH— or nitrogen, Y represents —CH₂—, —CH₂CH₂—, —CH═CH—, —CH₂—O—, or —O—CH₂—, Rₘ and Rₙ each represents a hydrogen atom, and Rₒ, Rₚ and R_{q} each independently represents a hydrogen atom, a hydroxyl group, a halogen atom, a (C6-C10)aryl group, a halo(C1-C3)alkyl group, a (C1-C6)alkoxy group, a tri(C1-C4)alkylsilanoxy group, or a benzodioxolyl group, or alternatively Rₒ and Rₚ or Rₚ and R_{q} together represent —CH₂—CH═CH— or —CH═CH—CH═CH—.

β-keto ester, which is the compound of formula B used as a starting material, may be commercially available. Otherwise, when Y represents —CH₂—, —CH₂CH₂—, —CH₂—O—, or —O—CH₂—, β-keto ester may be prepared according to the method described in J. Org. Chem., Vol. 43, No. 10, 1978, 2087-2088, specifically by reacting a commercially available acyl chloride derivative with Meldrum's acid and heating the resulting product under reflux in the presence of an ethanol solvent. When Y represents —CH═CH—, the desired compound may be prepared by reacting a cinnamic acid derivative with carbonyldiimidazole (CDI) to activate the acid moiety of the cinnamic acid derivative with acyl imidazolide and reacting the activated compound with either ethyl acetate or ethyl thioacetate in the presence of lithium bis(trimethylsilyl)amide (LiHMDS).

2-hydrazinopyridine may preferably be used in an amount of 1.0 to 3 molar equivalents based on 1 molar equivalent of the compound of formula II.

The polar organic solvent may preferably be selected from C1-C4 alcohol such as methanol, ethanol, n-propanol, isopropanol, n-butanol or t-butanol, acetic acid, and a mixture thereof. Ethanol or acetic acid is more preferred.

The heating may preferably be carried out at a reflux temperature of the solvent, preferably at a temperature of about 100 to about 130° C., for example.

The reaction may preferably be carried out for 2 to 72 hours.

The present invention provides a pharmaceutical composition for preventing or treating cardiovascular diseases, comprising a compound selected from:
1-(pyridin-2-yl)-3-phenyl-4-propyl-1H-pyrazol-5-ol (211),
1-(pyridin-2-yl)-3-phenyl-4-benzyl-1H-pyrazol-5-ol (212),
1-(pyridin-2-yl)-3-phenyl-4-(4-nitrobenzyl)1H-pyrazol-5-ol (225),
1-(pyridin-2-yl)-3-phenyl-4-propyl-1H-pyrazol-5-ol hydrochloride (278),
1-(pyridin-2-yl)-3-phenyl-4-propyl-1H-5-(isopropyloxycarbonyloxymethyloxy)pyrazole (306),
1-(pyridin-2-yl)-3-[(3-methoxy-4-hydroxyphenyl)-E-ethenyl]-4-benzyl-1H-pyrazol-5-ol hydrochloride (308),
1-(pyridin-2-yl)-3-[(3-methoxy-4-hydroxyphenyl)-E-ethenyl]-4-benzyl-5-hydroxypyrazole (274),
3-(4-nitrophenyl)-3-[2-(pyrimidin-2-yl)hydrazono]propionic acid ethyl ester,
1-(pyrimidin-2-yl)-3-(4-nitrophenyl)-5-1H-pyrazol-5-ol (105),
1-(pyridin-2-yl)-3-(1,2-diphenyl-E-ethenyl)-1H-pyrazol-5-ol (263),
1-(pyridin-2-yl)-3-phenyl-1H-pyrazol-5-ol hydrochloride (305),
3-(4-methoxyphenylamino)-1H-pyrazol-5-ol (15),
4-hexylidene-3-phenyl-1-(pyridin-2-yl)-1H-pyrazol-5-ol (213),
1-(pyridin-2-yl)-3-(3-(trifluoromethyl)phenyl)-1H-pyrazol-5-ol,
1-(pyridin-2-yl)-3-(4-(trifluoromethyl)phenyl)-1H-pyrazol-5-ol,
3-(3-nitrophenyl)-1-(pyridin-2-yl)-1H-pyrazol-5-ol,
1-(pyridin-2-yl)-3-(3,4,5-trimethoxyphenyl)-1H-pyrazol-5-ol,
3-(2-fluorophenyl)-1-(pyridin-2-yl)-1H-pyrazol-5-ol,
3-(3-fluorophenyl)-1-(pyridin-2-yl)-1H-pyrazol-5-ol,
3-(4-fluorophenyl)-1-(pyridin-2-yl)-1H-pyrazol-5-ol,
3-(2-chlorophenyl)-1-(pyridin-2-yl)-1H-pyrazol-5-ol,
3-(3-chlorophenyl)-1-(pyridin-2-yl)-1H-pyrazol-5-ol,
3-(4-chlorophenyl)-1-(pyridin-2-yl)-1H-pyrazol-5-ol,
3-(3-bromophenyl)-1-(pyridin-2-yl)-1H-pyrazol-5-ol,
3-(4-bromophenyl)-1-(pyridin-2-yl)-1H-pyrazol-5-ol,
3-(2-iodophenyl)-1-(pyridin-2-yl)-1H-pyrazol-5-ol,
3-(3-iodophenyl)-1-(pyridin-2-yl)-1H-pyrazol-5-ol,
3-(4-iodophenyl)-1-(pyridin-2-yl)-1H-pyrazol-5-ol,
3-(4-iodophenyl)-1-(pyrimidin-2-yl)-1H-pyrazol-5-ol,
2-(3-(4-iodophenyl)-5-(triisopropylsilyloxy)-1H-pyrazol-1-yl)pyridine,
3-(4-bromophenyl)-1-(pyridin-2-yl)-1H-pyrazol-5-yl t-butyl carbonate,
t-butyl 3-(2-iodophenyl)-1-(pyridin-2-yl)-1H-pyrazol-5-yl carbonate,
t-butyl 3-(3-iodophenyl)-1-(pyridin-2-yl)-1H-pyrazol-5-yl carbonate,
t-butyl 3-(4-iodophenyl)-1-(pyridin-2-yl)-1H-pyrazol-5-yl carbonate,
t-butyl 3-(4-iodophenyl)-1-(pyrimidin-2-yl)-1H-pyrazol-5-yl carbonate,
3-(4-bromophenyl)-1-(pyridin-2-yl)-1H-pyrazol-5-yl acetate,
3-(biphenyl-4-yl)-1-(pyridin-2-yl)-1H-pyrazol-5-yl t-butyl carbonate,
3-(4-(benzo[d][1,3]dioxol-5-yl)phenyl)-1-(pyridin-2-yl)-1H-pyrazol-5-yl t-butyl carbonate,
3-(3′-phenylbiphenyl-4-yl)-1-(pyridin-2-yl)-1H-pyrazol-5-yl t-butyl carbonate,
t-butyl 3-(4-(naphthalene)phenyl)-1-(pyridin-2-yl)-1H-pyrazol-5-yl carbonate,
t-butyl 3-(3′-(dimethylamino)biphenyl)-1-(pyridin-2-yl)-1H-pyrazol-5-yl carbonate,
3-(2-(benzo[d][1,3]dioxol-5-yl)phenyl)-1-(pyridin-2-yl)-1H-pyrazol-5-yl t-butyl carbonate,
3-(3′-phenylbiphenyl-2-yl)-1-(pyridin-2-yl)-1H-pyrazol-5-yl t-butyl carbonate,
3-(biphenyl-3-yl)-1-(pyridin-2-yl)-1H-pyrazol-5-yl t-butyl carbonate,
3-(4′-(benzyloxy)biphenyl-3-yl)-1-(pyridin-2-yl)-1H-pyrazol-5-yl t-butyl carbonate,
3-(4′-bromobiphenyl-3-yl)-1-(pyridin-2-yl)-1H-pyrazol-5-yl t-butyl carbonate,
t-butyl 3-(3′-formylbiphenyl-3-yl)-1-(pyridin-2-yl)-1H-pyrazol-5-yl carbonate,
t-butyl 3-(2′-phenoxybiphenyl-3-yl)-1-(pyridin-2-yl)-1H-pyrazol-5-yl carbonate,
3-(3-(benzo[d][1,3]dioxol-5-yl)phenyl)-1-(pyridin-2-yl)-1H-pyrazol-5-yl t-butyl carbonate,
3-(3′-phenylbiphenyl-3-yl)-1-(pyridin-2-yl)-1H-pyrazol-5-yl t-butyl carbonate,
t-butyl 3-(3-(naphthalene-1-yl)phenyl)-1-(pyridin-2-yl)-1H-pyrazol-5-yl carbonate,
t-butyl 3-(3′-(dimethylamino)biphenyl-3-yl)-1-(pyridin-2-yl)-1H-pyrazol-5-yl carbonate,
t-butyl 3-(4′-methoxybiphenyl-4-yl)-1-(pyridin-2-yl)-1H-pyrazol-5-yl carbonate,
3-(4′-(benzyloxy)biphenyl-4-yl)-1-(pyridin-2-yl)-1H-pyrazol-5-yl t-butyl carbonate,
3-(4′-bromobiphenyl-4-yl)-1-(pyridin-2-yl)-1H-pyrazol-5-yl t-butyl carbonate,
3-(3′-formylbiphenyl-4-yl)-1-(pyridin-2-yl)-1H-pyrazol-5-yl t-butyl carbonate,
t-butyl 3-(2′-phenoxybiphenyl-4-yl)-1-(pyridin-2-yl)-1H-pyrazol-5-yl carbonate,
3-(biphenyl-4-yl)-1-(pyrimidin-2-yl)-1H-pyrazol-5-yl t-butyl carbonate,
3-(4-(benzo[d][1,3]dioxol-5-yl)phenyl)-1-(pyrimidin-2-yl)-1H-pyrazol-5-yl t-butyl carbonate,
3-(3′-phenylbiphenyl-4-yl)-1-(pyrimidin-2-yl)-1H-pyrazol-5-yl t-butyl carbonate,
2-(3-(4′-bromobiphenyl-4-yl)-5-(triisopropylsilyloxy)-1H-pyrazol-1-yl)pyridine,
2-(3-(biphenyl-4-yl)-5-(diphenylboryloxy)-1H-pyrazol-1-yl)pyridine,
3-(biphenyl-4-yl)-1-(pyridin-2-yl)-1H-pyrazol-5-ol,
3-(4-(benzo[d][1,3]dioxol-5-yl)phenyl)-1-(pyridin-2-yl)-1H-pyrazol-5-ol,
3-(3′-phenylbiphenyl-4-yl)-1-(pyridin-2-yl)-1H-pyrazol-5-ol,
3-(4-(naphthalene-1-yl)phenyl)-1-(pyridin-2-yl)-1H-pyrazol-5-ol,
3-(3′-(dimethylamino)biphenyl-4-yl)-1-(pyridin-2-yl)-1H-pyrazol-5-ol,
3-(2-(benzo[d][1,3]dioxol-5-yl)phenyl)-1-(pyridin-2-yl)-1H-pyrazol-5-ol,
3-(3′-phenylbiphenyl-2-yl)-1-(pyridin-2-yl)-1H-pyrazol-5-ol,
3-(biphenyl-3-yl)-1-(pyridin-2-yl)-1H-pyrazol-5-ol,
3-(4′-(benzyloxy)biphenyl-3-yl)-1-(pyridin-2-yl)-1H-pyrazol-5-ol,
3-(4′-bromobiphenyl-3-yl)-1-(pyridin-2-yl)-1H-pyrazol-5-ol,
3-(3′-formylbiphenyl-3-yl)-1-(pyridin-2-yl)-1H-pyrazol-5-ol,
3-(2′-phenoxybiphenyl-3-yl)-1-(pyridin-2-yl)-1H-pyrazol-5-ol,
3-(3-(benzo[d][1,3]dioxol-5-yl)phenyl)-1-(pyridin-2-yl)-1H-pyrazol-5-ol,
3-(3′-phenylbiphenyl-3-yl)-1-(pyridin-2-yl)-1H-pyrazol-5-ol,
3-(3-(naphthalene-1-yl)phenyl)-1-(pyridin-2-yl)-1H-pyrazol-5-ol,
3-(3′-(dimethylamino)biphenyl-3-yl)-1-(pyridin-2-yl)-1H-pyrazol-5-ol,
3-(4′-methoxybiphenyl-4-yl)-1-(pyridin-2-yl)-1H-pyrazol-5-ol,
3-(4′-(benzyloxy)biphenyl-4-yl)-1-(pyridin-2-yl)-1H-pyrazol-5-ol,
3-(4′-bromobiphenyl-4-yl)-1-(pyridin-2-yl)-1H-pyrazol-5-ol,
3-(3′-formylbiphenyl-4-yl)-1-(pyridin-2-yl)-1H-pyrazol-5-ol,
3-(2′-phenoxybiphenyl-4-yl)-1-(pyridin-2-yl)-1H-pyrazol-5-ol,
3-(biphenyl-4-yl)-1-(pyrimidin-2-yl)-1H-pyrazol-5-ol,
3-(4-(benzo[d][1,3]dioxol-5-yl)phenyl)-1-(pyrimidin-2-yl)-1H-pyrazol-5-ol,
3-(3′-phenylbiphenyl-4-yl)-1-(pyridin-2-yl)-1H-pyrazol-5-ol,
3-phenyl-4-propyl-1-(pyridin-2-yl)-1H-pyrazol-5-ol,
4-benzyl-3-phenyl-1-(pyridin-2-yl)-1H-pyrazol-5-ol,
3-benzyl-1-(pyridin-2-yl)-1H-pyrazol-5-ol,
3-(o-bromobenzyl)-1-(pyridin-2-yl)-1H-pyrazol-5-ol,
3-(p-methoxybenzyl)-1-(pyridin-2-yl)-1H-pyrazol-5-ol,
3-phenethyl-1-(pyridin-2-yl)-1H-pyrazol-5-ol,
3-(p-chlorobenzyl)-1-(pyridin-2-yl)-1H-pyrazol-5-ol,
3-(phenoxymethyl)-1-(pyridin-2-yl)-1H-pyrazol-5-ol,
3-(naphthalene-3-yloxy)methyl)-1-(pyridin-2-yl)-1H-pyrazol-5-ol,
3-((4-chlorophenoxy)methyl)-1-(pyridin-2-yl)-1H-pyrazol-5-ol,
3-((2,4-dichlorophenoxymethyl)-1-(pyridin-2-yl)-1H-pyrazol-5-ol,
1-(pyridin-2-yl)-3-styryl-1H-pyrazol-5-ol,
3-(4-methoxystyryl)-1-(pyridin-2-yl)-1H-pyrazol-5-ol,
3-(3,4-dimethoxystyryl)-1-(pyridin-2-yl)-1H-pyrazol-5-ol,
3-(3,4-dichlorostyryl)-1-(pyridin-2-yl)-1H-pyrazol-5-ol,
3-(4-i-propylstyryl)-1-(pyridin-2-yl)-1H-pyrazol-5-ol,
3-(4-trifluoromethylstyryl)-1-(pyridin-2-yl)-1H-pyrazol-5-ol,
3-(4-t-butyldimethylsilyloxystyryl)-1-(pyridin-2-yl)-1H-pyrazol-5-ol,
3-(3-methoxy-4-t-butyldimethylsilyloxystyryl)-1-(pyridin-2-yl)-1H-pyrazol-5-ol,
3-(3,5-dimethoxy-4-t-butyldimethylsilyloxystyryl)-1-(pyridin-2-yl)-1H-pyrazol-5-ol,
3-(3-methoxy-4-t-butyldimethylsilyloxystyryl)-1-(pyridin-2-yl)-1H-pyrazol-5-thiol,
3-(4-hydroxystyryl)-1-(pyridin-2-yl)-1H-pyrazol-5-ol,
3-(3-methoxy-4-hydroxystyryl)-1-(pyridin-2-yl)-1H-pyrazol-5-ol,
3-(3,5-dimethoxy-4-hydroxystyryl)-1-(pyridin-2-yl)-1H-pyrazol-5-ol,
3-(3-methoxy-4-hydroxystyryl)-1-(pyridin-2-yl)-1H-pyrazol-5-thiol, and
4-((E)-2-(5-amino-1-(pyridin-2-yl)-1H-pyrazol-3-yl)vinyl)-2-methoxyphenol;

or a pharmaceutically acceptable salt thereof.

As used herein, the term “pharmaceutically acceptable salt” refers to a salt commonly used in the pharmaceutical industry, and examples thereof may be a salt of inorganic acid prepared using hydrochloric acid, nitric acid, phosphoric acid, bromic acid, iodic acid, perchloric acid, tartaric acid, sulfuric acid, etc.; a salt of organic acid prepared using acetic acid, trifluoroacetic acid, citric acid, maleic acid, succinic acid, oxalic acid, benzoic acid, tartaric acid, fumaric acid, mandelic acid, propionic acid, citric acid, lactic acid, glycolic acid, gluconic acid, galacturonic acid, glutamic acid, glutaric acid, glucuronic acid, aspartic acid, ascorbic acid, carbonic acid, vanillic acid, hydroiodic acid, etc.; and a salt of sulfonic acid prepared using methanesulfonic acid, ethanesulfonic acid, benzenesulfonic acid, p-toluenesulfonic acid, naphthalene sulfonic acid, etc. However, the pharmaceutically acceptable salt of the present invention is not limited thereto. The pharmaceutically acceptable sale may preferably be a hydrochloric acid salt.

The composition of the present invention can effectively inhibit the migration of smooth muscle cells and further inhibit the generation of reactive oxygen species and thus can be effectively used for the treatment of cardiovascular diseases such as atherosclerosis and vascular restenosis. Moreover, the composition of the present invention has therapeutic effects on cardiovascular diseases similar to those of the existing pyrazole derivative with a proven therapeutic effect on atherosclerosis (see Korean Patent No. 10-0987557) and has lower toxicity and higher stability in blood, thus effectively treating cardiovascular diseases without side effects.

Therefore, the composition of the present invention is effective for the treatment of cardiovascular diseases, and particularly effective for the prevention and treatment of vascular restenosis. That is, the composition of the present invention can be used for the purpose of preventing and treating coronary artery stenosis causing myocardial infarction or angina. Moreover, the composition of the present invention can be used for the purpose of preventing restenosis that frequently occurs after percutaneous transluminal coronary angioplasty performed for treating coronary artery stenosis. Examples of the percutaneous transluminal coronary angioplasty include balloon angioplasty, stent implantation, coronary artery bypass graft, and venovenostomy. An example of the use of the pharmaceutical composition according to the present invention is to administer the pharmaceutical composition in the same manner as conventional medicines or coat the pharmaceutical composition on a stent. The pharmaceutical composition may preferably be used in a sustained drug-releasing stent.

In the present invention, the term “cardiovascular diseases” include heart diseases such as heart failure, hypertensive heart disease, arrhythmia, congenital heart disease, myocardial infarction, angina, etc., and vascular diseases such as stroke, peripheral vascular disease, etc., and the cardiovascular diseases may be ischemic cardiovascular diseases in a broad sense. In particular, the cardiovascular diseases may be arteriosclerosis or vascular restenosis.

The composition of the present invention may include pharmaceutically acceptable additives, such as a diluent, a binder, a disintegrant, a lubricant, a pH-adjusting agent, an antioxidant and a solubilizer, within the range where effects of the present invention are not impaired.

Examples of the diluent include sugar, starch, microcrystalline cellulose, lactose (lactose hydrate), glucose, D-mannitol, alginate, an alkaline earth metal salt, clay, polyethylene glycol, anhydrous dibasic calcium phosphate, and a mixture thereof; Examples of the binder include starch, microcrystalline cellulose, highly dispersive silica, mannitol, D-mannitol, sucrose, lactose hydrate, polyethylene glycol, polyvinylpyrrolidone (povidone), a polyvinylpyrrolidone copolymer (copovidone), hypromellose, hydroxypropylcellulose, natural gum, synthetic gum, copovidone, gelatin, and a mixture thereof.

Examples of the disintegrant include starches or modified starches such as sodium starch glycolate, corn starch, potato starch, and pregelatinized starch; clays such as bentonite, montmorillonite, and veegum; celluloses such as microcrystalline cellulose, hydroxypropylcellulose, and carboxymethylcellulose; algins such as sodium alginate, and alginic acid; crosslinked celluloses such as croscarmellose sodium; gums such as guar gum, and xanthan gum; crosslinked polymers such as crosslinked polyvinylpyrrolidone (crospovidone); effervescent agents such as sodium bicarbonate and citric acid, and mixtures thereof.

Examples of the lubricant include talc, stearic acid, magnesium stearate, calcium stearate, sodium lauryl sulfate, hydrogenated vegetable oil, sodium benzoate, sodium stearyl fumarate, glyceryl behenate, glyceryl monolaurate, glyceryl monostearate, glyceryl palmitostearate, colloidal silicon dioxide, and mixtures thereof.

Examples of the pH-adjusting agent include acidifying agents such as acetic acid, adipic acid, ascorbic acid, sodium ascorbate, sodium etherate, malic acid, succinic acid, tartaric acid, fumaric acid, and citric acid, and basifying agents such as precipitated calcium carbonate, aqueous ammonia, meglumine, sodium carbonate, magnesium oxide, magnesium carbonate, sodium citrate, and tribasic calcium phosphate.

Examples of the antioxidant include dibutyl hydroxy toluene, butylated hydroxyanisole, tocopherol acetate, tocopherol, propyl gallate, sodium hydrogen sulfite, and sodium pyrosulfite. Examples of the solubilizer that can be used in the immediate-release compartment of the present invention include sodium lauryl sulfate, polyoxyethylene sorbitan fatty acid ester (such as polysorbate), docusate sodium and poloxamer.

Moreover, In order to prepare a delayed-release formulation, the composition of the present invention may include an enteric polymer, a water-insoluble polymer, a hydrophobic compound, and a hydrophilic polymer.

The enteric polymer refers to a polymer which is insoluble or stable under acidic conditions of less than pH 5 and is dissolved or degraded under specific pH conditions of pH 5 or higher. For example, the enteric polymer may be enteric cellulose derivatives such as hypromellose acetate succinate, hypromellose phthalate (hydroxypropylmethylcellulose phthalate), hydroxymethylethylcellulose phthalate, cellulose acetate phthalate, cellulose acetate succinate, cellulose acetate maleate, cellulose benzoate phthalate, cellulose propionate phthalate, methylcellulose phthalate, carboxymethylethylcellulose, ethylhydroxyethylcellulose phthalate, and methylhydroxyethylcellulose; enteric acrylic acid copolymers such as a styrene/acrylic acid copolymer, a methyl acrylate/acrylic acid copolymer, a methyl acrylate/methacrylic acid copolymer (e.g., Acryl-EZE), a butyl acrylate/styrene/acrylic acid copolymer, and a methyl acrylate/methacrylic acid/octyl acrylate copolymer; enteric polymethacrylate copolymers such as a poly(methacrylic acid/methyl methacrylate) copolymer (e.g., Eudragit L or Eudragit S, manufactured by Evonik, Germany), and a poly(methacrylic acid/ethyl acrylate) copolymer (e.g., Eudragit L100-55); enteric maleic acid copolymers such as a vinyl acetate/maleic anhydride copolymer, a styrene/maleic anhydride copolymer, a styrene/maleic monoester copolymer, a vinyl methyl ether/maleic anhydride copolymer, an ethylene/maleic anhydride copolymer, a vinyl butyl ether/maleic anhydride copolymer, an acrylonitrile/methyl acrylate/maleic anhydride copolymer, and a butyl acrylate/styrene/maleic anhydride copolymer; and enteric polyvinyl derivatives such as polyvinyl alcohol phthalate, polyvinylacetal phthalate, polyvinylbutyrate phthalate, and polyvinylacetacetal phthalate.

The water-insoluble polymer refers to a pharmaceutically acceptable water-insoluble polymer which controls the release of a drug. For example, the water-insoluble polymer may be polyvinyl acetate (e.g. Kollicoat SR30D), a water-insoluble polymethacrylate copolymer {e.g. poly(ethyl acrylate-methyl methacrylate) copolymer (such as Eudragit NE30D), a poly(ethyl acrylate-methyl methacrylate-trimethylaminoethyl methacrylate) copolymer (e.g. Eudragit RSPO), etc.}, ethylcellulose, cellulose ester, cellulose ether, cellulose acylate, cellulose diacylate, cellulose triacylate, cellulose acetate, cellulose diacetate, cellulose triacetate, etc.

The hydrophobic compound refers to a pharmaceutically acceptable water-insoluble material which controls the release of a drug. For example, the hydrophobic compound may be fatty acids and fatty acid esters such as glyceryl palmitostearate, glyceryl stearate, glyceryl behenate, cetyl palmitate, glyceryl monooleate and stearic acid; fatty acid alcohols such as cetostearyl alcohol, cetyl alcohol and stearyl alcohol; waxes such as carnauba wax, beeswax and microcrystalline wax; and inorganic materials such as talc, precipitated calcium carbonate, calcium hydrogen phosphate, zinc oxide, titanium oxide, kaolin, bentonite, montmorillonite and veegum.

The hydrophilic polymer refers to a pharmaceutically acceptable water-soluble polymer which controls the release of a drug. For example, the hydrophilic polymer may be saccharides such as dextrin, polydextrin, dextran, pectin and a pectin derivative, alginate, polygalacturonic acid, xylan, arabinoxylan, arabinogalactan, starch, hydroxypropyl starch, amylose and amylopectin; cellulose derivatives such as hypromellose, hydroxypropylcellulose, hydroxymethylcellulose, hydroxyethylcellulose, methylcellulose, and sodium carboxymethylcellulose; gums such as guar gum, locust bean gum, tragacanth, carrageenan, gum acacia, gum arabic, gellan gum and xanthan gum; proteins such as gelatin, casein and zein; polyvinyl derivatives such as polyvinyl alcohol, polyvinylpyrrolidone and polyvinylacetal diethylaminoacetate; hydrophilic polymethacrylate copolymers such as a poly(butyl methacrylate-(2-dimethylaminoethyl)methacrylate-methyl methacrylate) copolymer (e.g. Eudragit E100, manufactured by Evonik, Germany), and a poly(ethyl acrylate-methyl methacrylate-triethylaminoethyl-methacrylate chloride) copolymer (e.g. Eudragit RL and RS, manufactured by Evonik, Germany); polyethylene derivatives such as polyethylene glycol, and polyethylene oxide; and carbomer.

In addition, the composition of the present invention may be formulated with the use of pharmaceutically acceptable additives such as various additives selected from colorants and fragrances.

In the present invention, the range of the additive that can be used in the present invention is not limited to the above-mentioned additives, the additive may be appropriately selected by those skilled in the art and the composition may be formulated with the use of the above-mentioned additives in a conventional dose.

The pharmaceutical composition in accordance with the present invention may be formulated into oral dosage forms such as powders, granules, tablets, capsules, suspensions, emulsions, syrups, and aerosols, medicines for external use, suppositories or sterile injection solutions, according to a conventional known method, and may be used. Moreover, the present invention provides a method for preventing or treating cardiovascular diseases, the method comprising administering to a subject including a mammal the composition comprising the compound represented by formula 1, formula I, or formula A, the above-mentioned compound, or a pharmaceutically acceptable salt thereof of the present invention. As used herein, the term “administering” means the introduction of the composition for preventing and treating cardiovascular diseases in accordance with the present invention to a patient by any appropriate method. The composition for preventing and treating cardiovascular diseases in accordance with the present invention may be administered via any conventional administration route as long as the composition can reach a target tissue. For example, the composition may be administered orally, intraperitoneally, intravenously, intramuscularly, subcutaneously, intradermally, intranasally, intrapulmonary, rectally, intracavitary, or intrathecally, but not limited thereto.

The composition for preventing or treating cardiovascular diseases in accordance with the present invention may be administered once a day or may be administered at regular time intervals twice or more a day.

The dosage of the compound represented by formula 1, formula I, or formula A, the above-mentioned compound, or a pharmaceutically acceptable salt thereof of the present invention varies depending on body weight, age, gender, and health state of the patient, diet, administration timing, administration route, excretion rate, severity of the disease, etc. Suitable dosages may be 0.1 to 100 mg/kg/day, more preferably 10 to 40 mg/kg/day, but may vary depending on the patient's severity, age, sex, etc.

Moreover, the present invention provides a drug delivery system for topical administration of the composition comprising the compound represented by formula 1, formula I, or formula A for preventing or treating vascular restenosis. The drug delivery system for topical administration may preferably be an implantable stent coated with the composition of the present invention.

As used herein, the term “stent” means a general device intended for endoluminal application, for example, application to a blood vessel and refers to a cylindrical medical device which is inserted into a narrowed or blocked blood vessel under X-ray irradiation to normalize the blood flow without surgical laparotomy when disorders of blood flow occur due to diseases in an area where blood is required to smoothly flow. For example, a vascular stent is described in the work “Textbook of Interventional Cardiology” by Eric J. Topol, Saunders Company, 1994. The stent may preferably be a sustained drug-releasing stent.

As a specific example, in order to prepare the stent according to the present invention, the above-mentioned composition may be coated on the stent.

Any suitable coating method known to those skilled in the art can be used to coat the composition on the stent. Examples of the coating method include a dip-coating method and a polymer-coating method. The dip-coating method is the simplest method because only the pharmaceutical composition is coated and thus facilitates the observation of biological effects of only the pharmaceutical composition, but not limited thereto. The stent of the present invention may preferably be prepared by coating a mixture of the composition and a polymeric material on a drug-releasing stent such that the composition according to the present invention can be slowly released. Examples of the polymeric materials that can be used in the drug-releasing stent are widely known in the art and may include polyurethane, polyethylene terephthalate, PLLA-poly-glycolic acid (PLGA) copolymer, polycaprolactone, poly-(hydroxybutyrate/hydroxyvalerate) copolymer, polyvinylpyrrolidone, polytetrafluoroethylene, poly(2-hydroxyethylmethacrylate), poly(etherurethane urea), silicone, acrylic, epoxide, polyester, urethane, parlene, polyphosphazene polymer, fluoropolymer, polyamide, polyolefin, and a mixture thereof, but not limited thereto.

Moreover, the present invention provides a method for removing vascular stenosis, the method comprising administering to a subject in need thereof a composition comprising a compound represented by formula 1, formula I, or formula A.

The present invention provides a method for removing vascular stenosis, the method comprising administering to a subject in need thereof a composition for preventing or treating cardiovascular diseases, comprising a compound represented by the following formula 1:

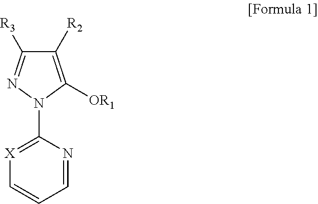

wherein X represents —CH— or nitrogen,

R₁ represents a hydrogen atom or an isopropyloxycarbonyloxymethyl,

R₂ represents a hydrogen atom, a C1-C4 linear or branched alkyl, or a substituted or unsubstituted benzyl, and

R₃ represents a phenyl, a nitrophenyl, a substituted or unsubstituted phenylethenyl, or a substituted or unsubstituted diphenylethenyl,

wherein when X represents —CH— and R₁ and R₂ each represents a hydrogen atom, R₃ represents a substituted or unsubstituted diphenylethenyl, or when X represents a nitrogen atom and R₁ and R₂ each represents a hydrogen atom, R₃ represents a nitrophenyl or a substituted or unsubstituted diphenylethenyl; and

wherein the substituent is a nitro, a hydroxyl, or a methoxyl.

The present invention provides a method for removing vascular stenosis, the method comprising administering to a subject in need thereof a composition for preventing or treating cardiovascular diseases, comprising a pharmaceutically acceptable salt of a compound represented by the following formula 1:

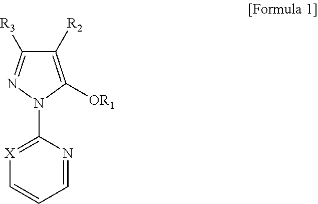

wherein X represents —CH— or nitrogen;

R₁ represents a hydrogen atom or an isopropyloxycarbonyloxymethyl;

R₂ represents a hydrogen atom, a C1-C4 linear or branched alkyl, or a substituted or unsubstituted benzyl; and

R₃ represents a phenyl, a nitrophenyl, a substituted or unsubstituted phenylethenyl, or a substituted or unsubstituted diphenylethenyl;

wherein when R₁ and R₂ each represents a hydrogen atom, R₃ represents a phenyl group, a nitrophenyl group, or a substituted or unsubstituted diphenylethenyl group; and

wherein the substituent is a nitro, a hydroxyl, or a methoxyl.

The present invention provides a method for removing vascular stenosis, the method comprising administering to a subject in need thereof a composition for preventing or treating cardiovascular diseases, comprising a compound represented by the following formula I or a pharmaceutically acceptable salt thereof:

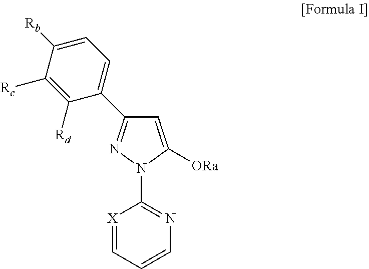

wherein X represents —CH— or nitrogen;

Rₐ represents a hydrogen atom, an acetyl group, a tri(C1-C4)alkylsilanyl group, a diphenylboranyl group, or a (t-butoxy)carbonyl group; and

R_{b}, R_{c} and R_{d} each independently represents a hydrogen atom, a halogen atom, a halo(C1-C3)alkyl group, a (C1-C6)alkoxy group, a benzo[d][1,3]dioxol group, a substituted or unsubstituted biphenyl group, or a substituted or unsubstituted (C6-C10)aryl group, and

wherein the substituent is a halogen atom, a (C1-C4)alkyl amine group, a halo(C1-C3)alkyl group, a (C1-C6)alkoxy group, a phenoxy group, a benzyloxy group, a formyl group, or a halogen-substituted phenyl group, with the proviso that R_{b}, R_{c} and R_{d} are not a hydrogen atom at the same time.

The present invention provides a method for removing vascular stenosis, the method comprising administering to a subject in need thereof a composition for preventing or treating cardiovascular diseases, comprising a compound represented by the following formula A or a pharmaceutically acceptable salt thereof:

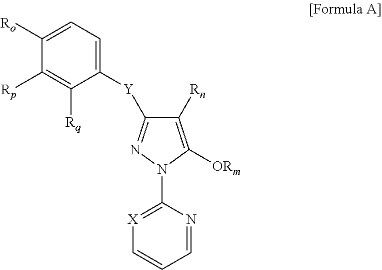

wherein X represents —CH— or nitrogen;

Y represents —CH₂—, —CH₂CH₂—, —CH═CH—, —CH₂—O—, or —O—CH₂—;

Rₘ represents a hydrogen atom, an acetyl group, a tri(C1-C4)alkylsilanyl group, a diarylboranyl group, or a (t-butoxy)carbamyl group;

Rₐ represents a hydrogen atom or a (C1-C4)alkyl group; and

Rₒ, Rₚ and R_{q} each independently represents a hydrogen atom, a hydroxyl group, a halogen atom, a (C6-C10)aryl group, a halo(C1-C3)alkyl group, a (C1-C6)alkoxy group, a tri(C1-C4)alkylsilanoxy group, or a benzodioxolyl group, or alternatively Rₒ and Rₚ or Rₚ and R_{q} together represent —CH₂—CH═CH—, —CH═CH—CH═CH—, or —CH═CH—CH═CH—CH₂—.

The present invention provides a method for removing vascular stenosis, the method comprising administering to a subject in need thereof a composition for preventing or treating cardiovascular diseases, comprising at least one compound selected from the group consisting of:
1-(pyridin-2-yl)-3-phenyl-4-propyl-1H-pyrazol-5-ol (211),
1-(pyridin-2-yl)-3-phenyl-4-benzyl-1H-pyrazol-5-ol (212),
1-(pyridin-2-yl)-3-phenyl-4-(4-nitrobenzyl)1H-pyrazol-5-ol (225),
1-(pyridin-2-yl)-3-phenyl-4-propyl-1H-pyrazol-5-ol hydrochloride (278),
1-(pyridin-2-yl)-3-phenyl-4-propyl-1H-5-(isopropyloxycarbonyloxymethyloxy)pyrazole (306),
1-(pyridin-2-yl)-3-[(3-methoxy-4-hydroxyphenyl)-E-ethenyl]-4-benzyl-1H-pyrazol-5-ol hydrochloride (308),
1-(pyridin-2-yl)-3-[(3-methoxy-4-hydroxyphenyl)-E-ethenyl]-4-benzyl-5-hydroxypyrazole (274),
3-(4-nitrophenyl)-3-[2-(pyrimidin-2-yl)hydrazono]propionic acid ethyl ester,
1-(pyrimidin-2-yl)-3-(4-nitrophenyl)-5-1H-pyrazol-5-ol (105),
1-(pyridin-2-yl)-3-(1,2-diphenyl-E-ethenyl)-1H-pyrazol-5-ol (263),
1-(pyridin-2-yl)-3-phenyl-1H-pyrazol-5-ol hydrochloride (305),
3-(4-methoxyphenylamino)-1H-pyrazol-5-ol (15),
4-hexylidene-3-phenyl-1-(pyridin-2-yl)-1H-pyrazol-5-ol (213),
1-(pyridin-2-yl)-3-(3-(trifluoromethyl)phenyl)-1H-pyrazol-5-ol,
1-(pyridin-2-yl)-3-(4-(trifluoromethyl)phenyl)-1H-pyrazol-5-ol,
3-(3-nitrophenyl)-1-(pyridin-2-yl)-1H-pyrazol-5-ol,
3-(4-nitrophenyl)-1-(pyridin-2-yl)-1H-pyrazol-5-ol,
3-(4-methoxyphenyl)-1-(pyridin-2-yl)-1H-pyrazol-5-ol,
3-(3,4-dimethoxyphenyl)-1-(pyridin-2-yl)-1H-pyrazol-5-ol,
1-(pyridin-2-yl)-3-(3,4,5-trimethoxyphenyl)-1H-pyrazol-5-ol,
3-(2-fluorophenyl)-1-(pyridin-2-yl)-1H-pyrazol-5-ol,
3-(3-fluorophenyl)-1-(pyridin-2-yl)-1H-pyrazol-5-ol,
3-(4-fluorophenyl)-1-(pyridin-2-yl)-1H-pyrazol-5-ol,
3-(2-chlorophenyl)-1-(pyridin-2-yl)-1H-pyrazol-5-ol,
3-(3-chlorophenyl)-1-(pyridin-2-yl)-1H-pyrazol-5-ol,
3-(4-chlorophenyl)-1-(pyridin-2-yl)-1H-pyrazol-5-ol,
3-(3-bromophenyl)-1-(pyridin-2-yl)-1H-pyrazol-5-ol,
3-(4-bromophenyl)-1-(pyridin-2-yl)-1H-pyrazol-5-ol,
3-(2-iodophenyl)-1-(pyridin-2-yl)-1H-pyrazol-5-ol,
3-(3-iodophenyl)-1-(pyridin-2-yl)-1H-pyrazol-5-ol,
3-(4-iodophenyl)-1-(pyridin-2-yl)-1H-pyrazol-5-ol,
3-(4-iodophenyl)-1-(pyrimidin-2-yl)-1H-pyrazol-5-ol,
2-(3-(4-iodophenyl)-5-(triisopropylsilyloxy)-1H-pyrazol-1-yl)pyridine,
3-(4-bromophenyl)-1-(pyridin-2-yl)-1H-pyrazol-5-yl t-butyl carbonate,
t-butyl 3-(2-iodophenyl)-1-(pyridin-2-yl)-1H-pyrazol-5-yl carbonate,
t-butyl 3-(3-iodophenyl)-1-(pyridin-2-yl)-1H-pyrazol-5-yl carbonate,
t-butyl 3-(4-iodophenyl)-1-(pyridin-2-yl)-1H-pyrazol-5-yl carbonate,
t-butyl 3-(4-iodophenyl)-1-(pyrimidin-2-yl)-1H-pyrazol-5-yl carbonate,
3-(4-bromophenyl)-1-(pyridin-2-yl)-1H-pyrazol-5-yl acetate,
3-(biphenyl-4-yl)-1-(pyridin-2-yl)-1H-pyrazol-5-yl t-butyl carbonate,
3-(4-(benzo[d][1,3]dioxol-5-yl)phenyl)-1-(pyridin-2-yl)-1H-pyrazol-5-yl t-butyl carbonate,
3-(3′-phenylbiphenyl-4-yl)-1-(pyridin-2-yl)-1H-pyrazol-5-yl t-butyl carbonate,
t-butyl 3-(4-(naphthalene)phenyl)-1-(pyridin-2-yl)-1H-pyrazol-5-yl carbonate,
t-butyl 3-(3′-(dimethylamino)biphenyl)-1-(pyridin-2-yl)-1H-pyrazol-5-yl carbonate,
3-(2-(benzo[d][1,3]dioxol-5-yl)phenyl)-1-(pyridin-2-yl)-1H-pyrazol-5-yl t-butyl carbonate,
3-(3′-phenylbiphenyl-2-yl)-1-(pyridin-2-yl)-1H-pyrazol-5-yl t-butyl carbonate,
3-(biphenyl-3-yl)-1-(pyridin-2-yl)-1H-pyrazol-5-yl t-butyl carbonate,
3-(4′-(benzyloxy)biphenyl-3-yl)-1-(pyridin-2-yl)-1H-pyrazol-5-yl t-butyl carbonate,
3-(4′-bromobiphenyl-3-yl)-1-(pyridin-2-yl)-1H-pyrazol-5-yl t-butyl carbonate,
t-butyl 3-(3′-formylbiphenyl-3-yl)-1-(pyridin-2-yl)-1H-pyrazol-5-yl carbonate,
t-butyl 3-(2′-phenoxybiphenyl-3-yl)-1-(pyridin-2-yl)-1H-pyrazol-5-yl carbonate,
3-(3-(benzo[d][1,3]dioxol-5-yl)phenyl)-1-(pyridin-2-yl)-1H-pyrazol-5-yl t-butyl carbonate,
3-(3′-phenylbiphenyl-3-yl)-1-(pyridin-2-yl)-1H-pyrazol-5-yl t-butyl carbonate,
t-butyl 3-(3-(naphthalene-1-yl)phenyl)-1-(pyridin-2-yl)-1H-pyrazol-5-yl carbonate,
t-butyl 3-(3′-(dimethylamino)biphenyl-3-yl)-1-(pyridin-2-yl)-1H-pyrazol-5-yl carbonate,
t-butyl 3-(4′-methoxybiphenyl-4-yl)-1-(pyridin-2-yl)-1H-pyrazol-5-yl carbonate,
3-(4′-(benzyloxy)biphenyl-4-yl)-1-(pyridin-2-yl)-1H-pyrazol-5-yl t-butyl carbonate,
3-(4′-bromobiphenyl-4-yl)-1-(pyridin-2-yl)-1H-pyrazol-5-yl t-butyl carbonate,
3-(3′-formylbiphenyl-4-yl)-1-(pyridin-2-yl)-1H-pyrazol-5-yl t-butyl carbonate,
t-butyl 3-(2′-phenoxybiphenyl-4-yl)-1-(pyridin-2-yl)-1H-pyrazol-5-yl carbonate,
3-(biphenyl-4-yl)-1-(pyrimidin-2-yl)-1H-pyrazol-5-yl t-butyl carbonate,
3-(4-(benzo[d][1,3]dioxol-5-yl)phenyl)-1-(pyrimidin-2-yl)-1H-pyrazol-5-yl t-butyl carbonate,
3-(3′-phenylbiphenyl-4-yl)-1-(pyrimidin-2-yl)-1H-pyrazol-5-yl t-butyl carbonate,
2-(3-(4′-bromobiphenyl-4-yl)-5-(triisopropylsilyloxy)-1H-pyrazol-1-yl)pyridine,
2-(3-(biphenyl-4-yl)-5-(diphenylboryloxy)-1H-pyrazol-1-yl)pyridine,
3-(biphenyl-4-yl)-1-(pyridin-2-yl)-1H-pyrazol-5-ol,
3-(4-(benzo[d][1,3]dioxol-5-yl)phenyl)-1-(pyridin-2-yl)-1H-pyrazol-5-ol,
3-(3′-phenylbiphenyl-4-yl)-1-(pyridin-2-yl)-1H-pyrazol-5-ol,
3-(4-(naphthalene-1-yl)phenyl)-1-(pyridin-2-yl)-1H-pyrazol-5-ol,
3-(3′-(dimethylamino)biphenyl-4-yl)-1-(pyridin-2-yl)-1H-pyrazol-5-ol,
3-(2-(benzo[d][1,3]dioxol-5-yl)phenyl)-1-(pyridin-2-yl)-1H-pyrazol-5-ol,
3-(3′-phenylbiphenyl-2-yl)-1-(pyridin-2-yl)-1H-pyrazol-5-ol,
3-(biphenyl-3-yl)-1-(pyridin-2-yl)-1H-pyrazol-5-ol,
3-(4′-(benzyloxy)biphenyl-3-yl)-1-(pyridin-2-yl)-1H-pyrazol-5-ol,
3-(4′-bromobiphenyl-3-yl)-1-(pyridin-2-yl)-1H-pyrazol-5-ol,
3′-(3-formylbiphenyl-3-yl)-1-(pyridin-2-yl)-1H-pyrazol-5-ol,
3-(2′-phenoxybiphenyl-3-yl)-1-(pyridin-2-yl)-1H-pyrazol-5-ol,
3-(3-(benzo[d][1,3]dioxol-5-yl)phenyl)-1-(pyridin-2-yl)-1H-pyrazol-5-ol,
3-(3′-phenylbiphenyl-3-yl)-1-(pyridin-2-yl)-1H-pyrazol-5-ol,
3-(3-(naphthalene-1-yl)phenyl)-1-(pyridin-2-yl)-1H-pyrazol-5-ol,
3-(3′-(dimethylamino)biphenyl-3-yl)-1-(pyridin-2-yl)-1H-pyrazol-5-ol,
3-(4′-methoxybiphenyl-4-yl)-1-(pyridin-2-yl)-1H-pyrazol-5-ol,
3-(4′-(benzyloxy)biphenyl-4-yl)-1-(pyridin-2-yl)-1H-pyrazol-5-ol,
3-(4′-bromobiphenyl-4-yl)-1-(pyridin-2-yl)-1H-pyrazol-5-ol,
3-(3′-formylbiphenyl-4-yl)-1-(pyridin-2-yl)-1H-pyrazol-5-ol,
3-(2′-phenoxybiphenyl-4-yl)-1-(pyridin-2-yl)-1H-pyrazol-5-ol,
3-(biphenyl-4-yl)-1-(pyrimidin-2-yl)-1H-pyrazol-5-ol,
3-(4-(benzo[d][1,3]dioxol-5-yl)phenyl)-1-(pyrimidin-2-yl)-1H-pyrazol-5-ol,
3-(3′-phenylbiphenyl-4-yl)-1-(pyridin-2-yl)-1H-pyrazol-5-ol,
3-phenyl-4-propyl-1-(pyridin-2-yl)-1H-pyrazol-5-ol,
4-benzyl-3-phenyl-1-(pyridin-2-yl)-1H-pyrazol-5-ol,
3-benzyl-1-(pyridin-2-yl)-1H-pyrazol-5-ol,
3-(o-bromobenzyl)-1-(pyridin-2-yl)-1H-pyrazol-5-ol,
3-(p-methoxybenzyl)-1-(pyridin-2-yl)-1H-pyrazol-5-ol,
3-phenethyl-1-(pyridin-2-yl)-1H-pyrazol-5-ol,
3-(p-chlorobenzyl)-1-(pyridin-2-yl)-1H-pyrazol-5-ol,
3-(phenoxymethyl)-1-(pyridin-2-yl)-1H-pyrazol-5-ol,
3-(naphthalene-3-yloxy)methyl)-1-(pyridin-2-yl)-1H-pyrazol-5-ol,
3-((4-chlorophenoxy)methyl)-1-(pyridin-2-yl)-1H-pyrazol-5-ol,
3-((2,4-dichlorophenoxymethyl)-1-(pyridin-2-yl)-1H-pyrazol-5-ol,
1-(pyridin-2-yl)-3-styryl-1H-pyrazol-5-ol,
3-(4-methoxystyryl)-1-(pyridin-2-yl)-1H-pyrazol-5-ol,
3-(3,4-dimethoxystyryl)-1-(pyridin-2-yl)-1H-pyrazol-5-ol,
3-(3,4-dichlorostyryl)-1-(pyridin-2-yl)-1H-pyrazol-5-ol,
3-(4-i-propylstyryl)-1-(pyridin-2-yl)-1H-pyrazol-5-ol,
3-(4-trifluoromethylstyryl)-1-(pyridin-2-yl)-1H-pyrazol-5-ol,
3-(4-t-butyldimethylsilyloxystyryl)-1-(pyridin-2-yl)-1H-pyrazol-5-ol,
3-(3-methoxy-4-t-butyldimethylsilyloxystyryl)-1-(pyridin-2-yl)-1H-pyrazol-5-ol,
3-(3,5-dimethoxy-4-t-butyldimethylsilyloxystyryl)-1-(pyridin-2-yl)-1H-pyrazol-5-ol,
3-(3-methoxy-4-t-butyldimethylsilyloxystyryl)-1-(pyridin-2-yl)-1H-pyrazol-5-thiol,
3-(4-hydroxystyryl)-1-(pyridin-2-yl)-1H-pyrazol-5-ol,
3-(3-methoxy-4-hydroxystyryl)-1-(pyridin-2-yl)-1H-pyrazol-5-ol,
3-(3,5-dimethoxy-4-hydroxystyryl)-1-(pyridin-2-yl)-1H-pyrazol-5-ol,
3-(3-methoxy-4-hydroxystyryl)-1-(pyridin-2-yl)-1H-pyrazol-5-thiol, and
4-((E)-2-(5-amino-1-(pyridin-2-yl)-1H-pyrazol-3-yl)vinyl)-2-methoxyphenol;

or a pharmaceutically acceptable salt thereof.

The present invention provides a method for removing vascular stenosis, the method comprising administering to a subject in need thereof a composition for preventing or treating cardiovascular diseases, comprising at least one compound selected from the group consisting of:
1-(pyridin-2-yl)-3-phenyl-4-propyl-1H-pyrazol-5-ol hydrochloride (278),
1-(pyridin-2-yl)-3-phenyl-1H-pyrazol-5-ol hydrochloride (305),
1-(pyridin-2-yl)-3-phenyl-4-propyl-1H-5-(isopropyloxycarbonyloxymethyloxy)pyrazole (306), and
1-(pyridin-2-yl)-3-[(3-methoxy-4-hydroxyphenyl)-E-ethenyl]-4-benzyl-1H-pyrazol-5-ol hydrochloride (308);

or a pharmaceutically acceptable salt thereof.

The method for removing vascular stenosis of the present comprises administering a pharmaceutically effective amount of a composition comprising a compound of formula 1, formula I, or formula A. It will be apparent to those skilled in the art that the suitable total daily dose may be determined by an attending physician within the scope of sound medical judgment. The specific therapeutically effective dose level for any particular patient may preferably vary depending on a variety of factors, including the kind and degree of a desired reaction, the specific composition, including the use of any other agents according to the intended use, the patient's age, weight, general health, gender, and diet, the time of administration, route of administration, and rate of the excretion of the composition, the duration of the treatment, other drugs used in combination or coincidentally with the specific composition, and like factors well known in the medical arts.

In a preferred aspect of the present invention, the treatment method of the present invention may comprise administering a pharmaceutical composition comprising a compound of formula 1, formula I, or formula A in combination with one or more known therapeutic agents.

Examples of the known therapeutic agent may include paclitaxel, sirolimus, etc., and the pharmaceutically effective amount of the therapeutic agent is known in the art and may be decided by an attending physician based on various conditions such as the severity of disease, combined administration with the composition of the present invention, etc. The combined administration of the composition of the present invention with the known therapeutic agent can alleviate side effects caused by the known therapeutic agent and may also result in synergistic therapeutic effects. In some cases, the known therapeutic agents may be administered as co-formulation or administered simultaneously with the composition of the present invention or the known therapeutic agent and the composition of the present invention may be administered at different time points.

Moreover, the present invention provides a use of the prevention or treatment of cardiovascular diseases of the composition comprising the compound represented formula 1, formula I, or formula A, the above-mentioned compound, or a pharmaceutically acceptable salt thereof, for preparing a pharmaceutical composition for the prevention or treatment of cardiovascular diseases.

Furthermore, the present invention provides a use in the prevention or treatment of cardiovascular diseases of the compound represented formula 1, formula I, or formula A, the above-mentioned compound, or a pharmaceutically acceptable salt thereof, for preparing a pharmaceutical composition for the prevention or treatment of vascular restenosis.

### Advantageous Effects

The compounds of the present invention have excellent inhibitory activity on the generation of reactive oxygen species and can be used for the treatment or prevention of cardiovascular diseases without any special side effects of conventional therapeutic agents.

### DESCRIPTION OF DRAWINGS

FIG. 1 is a view showing the migration degree of smooth muscle cells in experimental groups and control groups observed under a microscope.

FIG. 2 is a graph showing the comparison of the migration degree of smooth muscle cells in experimental groups and control groups.

FIG. 3 is a view showing the human aortic smooth muscle cells in experimental groups and control groups observed under a confocal microscope.

FIG. 4 is a graph showing the relative fluorescence intensity of human aortic smooth muscle cells in experimental groups and control groups.

FIGS. 5 to 10 are respectively graphs showing the relative superoxide generation calculated from Nox1/Nox2/Nox3/Nox4/Duox1/Duox2 membranes in experimental groups and control groups.

### MODE FOR INVENTION

Hereinafter, the present invention will be described in more detail with reference to the following Examples and Experimental Examples. However, it should be understood that the following examples and experimental examples are provided only for illustrating the present invention and should not be construed as limiting the scope and spirit of the present invention.

Reagents and solvents mentioned below were those available from Sigma-Aldrich Korea, Alfa Aesar, or Tokyo Chemical Industry (TCI). Moreover, ¹H-NMR and ¹³C-NMR spectra were obtained with a JEOL Eclipse FT 300 MHz Spectrometer and mass spectra was obtained measured with a JEOL MStation JMS 700 mass Spectrometer.

### Example 1

### Synthesis of 1-(pyridin-2-yl)-3-phenyl-4-propyl-1H-pyrazol-5-ol.HCl (278)

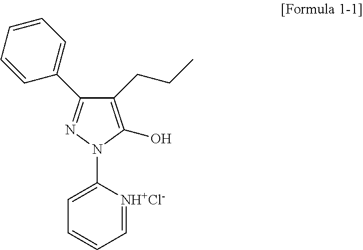

1-(pyridin-2-yl)-3-phenyl-4-propyl-1H-pyrazol-5-ol (280 mg) prepared in Example 1 was dissolved in ethyl ether (4 mL) in a round-bottom flask. Ethyl ester (0.55 mL) in which 2 M HCl was dissolved was slowly added dropwise at 0° C. The solvent was removed by vacuum filtration, and the resulting solid was washed with hexane and ethyl acetate and dried under vacuum to give the target compound (270 mg).

¹H NMR (300 MHz, DMSO-d₆) δ 8.44 (1H, d, J=4.2 Hz), 8.08-8.03 (2H, m), 7.66-7.64 (2H, m), 7.48-7.42 (3H, m), 7.34-7.30 (1H, m), 2.49 (2H, brs), 2.43 (2H, t, J=7.5 Hz), 1.48 (2H, m), 0.48 (3H, t, J=7.3 Hz).

### Example 2

### 1-(pyridin-2-yl)-3-phenyl-1H-pyrazol-5-ol.HCl (305)

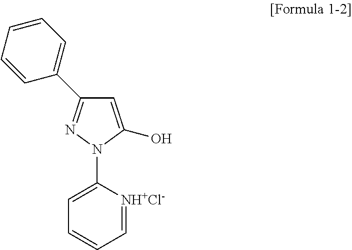

Ethyl benzoylacetate (1.92 g, 9.99 mmol) and ethanol (10 mL) were placed in a round-bottom flask, and 2-hydrazinopyridine (1.1 g, 10.0 mmol) diluted with ethanol (10 mL) was slowly added dropwise at 0° C. The resulting solution was heated under reflux at 100° C. for 8 hours. The solvent was removed by distillation under reduced pressure, and the resulting solid was washed with hexane and ethyl acetate and dried under vacuum to give 1-(pyridin-2-yl)-3-phenyl-1H-pyrazol-5-ol in 87% yield.

Ethyl ether (4 mL) was added to the prepared 1-(pyridin-2-yl)-3-phenyl-1H-pyrazol-5-ol (237 mg) in a round-bottom flask, and ethyl ether (0.55 mL) in which 2 M HCl was dissolved was slowly added dropwise at 0° C. The solvent was removed by vacuum filtration, and the resulting solid was washed with hexane and ethyl acetate and dried under vacuum to give the target compound in 87% yield.

¹H NMR (300 MHz, DMSO-d₆) δ 8.48-8.46 (m, 1H), 8.08 (t, 1H, J=8.3 Hz), 7.96 (d, 1H, J=8.4 Hz), 7.87 (d, 2H, J=8.3 Hz), 7.46-7.35 (m, 4H), 6.64 (br, 4H), 6.14 (s, 1H).

### Example 3

### 1-(pyridin-2-yl)-3-phenyl-4-propyl-1H-5-(isopropyloxycarbonyloxymethyloxy)pyrazole (306)

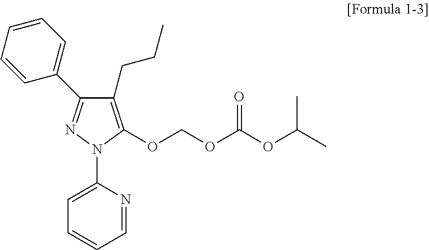 [SMILES: CCCc1c(-c2ccccc2)nn(-c2ccccn2)c1OCOC(=O)OC(C)C]

1-(pyridin-2-yl)-3-phenyl-4-propyl-1H-pyrazol-5-ol (438 mg) prepared in Example 1, K₂CO₃ (650 mg), and Bu₄NHSO₄ (532 mg) were added to a mixed solvent of water (8 mL) and dichloromethane (8 mL), and a solution in which isopropyloxycarbonyloxymethyliodide (497 mg) was dissolved in dichloromethane (2 mL) was added thereto. Then, the resulting solution was vigorously stirred overnight such that the compounds were reacted. The organic layer obtained by extracting the resulting solution with dichloromethane was washed with water and brine and concentrated. The resulting residue was purified by column chromatography (developing solvent: hexane/ethyl acetate (EtOAc)=10/1) to give the target compound (361 mg, 58% yield).

¹H NMR (300 MHz, CDCl₃) δ 8.48-8.46 (1H, m), 7.89 (1H, d, J=8.2 Hz), 7.80 (1H, m), 7.78-7.68 (2H, m), 7.57-7.34 (3H, m), 7.23-7.16 (1H, m), 5.84 (2H, s), 4.86 (1H, m), 2.55 (2H, d, J=7.7 Hz), 1.58 (2H, m), 1.25 (6H, d, J=6.3 Hz), 0.91 (3H, t, J=7.5 Hz)

¹³C NMR (75 MHz, CDCl₃) δ 153.6, 152.0, 150.8, 149.3, 147.9, 138.3, 134.0, 128.4, 128.0, 127.6, 121.3, 115.9, 110.0, 92.6, 72.7, 24.7, 22.9, 21.6, 14.1.

### Example 4

### 1-(pyridin-2-yl)-3-[(3-methoxy-4-hydroxyphenyl)-E-ethenyl]-4-benzyl-1H-pyrazol-5-ol.HCl (308)

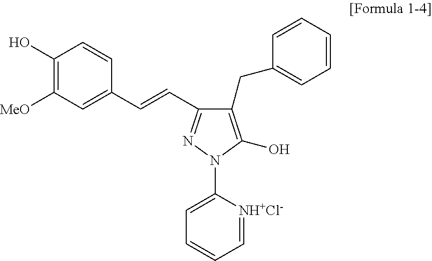

2-benzyl-3-oxo-3-[(3-methoxy-4-hydroxyphenyl)-E-ethenyl]-propionic acid ethyl ester (3.55 g, 10 mmol) and acetic acid (10 ml) were placed in a round-bottom flask, and 2-hydrazinopyridine (1.1 g, 10.1 mmol) diluted with acetic acid (3 mL) was slowly added dropwise at 0° C. The resulting solution was heated under reflux at 100° C. for 2 days. The solvent was removed by distillation under reduced pressure, and the residue was purified by column chromatography (developing solvent: hexane/ethyl acetate (EtOAc)=15/1) to give 1-(pyridin-2-yl)-3-[(3-methoxy-4-hydroxyphenyl)-E-ethenyl]-4-benzyl-1H-pyrazol-5-ol (1.32 g).

The obtained 1-(pyridin-2-yl)-3-[(3-methoxy-4-hydroxyphenyl)-E-ethenyl]-4-benzyl-1H-pyrazol-5-ol (1.10 g) was dissolved in ethyl ether (15 mL) in a round-bottom flask, and ethyl ether (1.5 mL) in which 2 M HCl was dissolved was slowly added dropwise at 0° C. The resulting solution was stirred at 40° C. for 24 hours, the solvent was removed by vacuum filtration, and then the resulting solid was washed with hexane and ethyl acetate and dried under vacuum to give the target compound (1.1 g).

¹H NMR (300 MHz, DMSO-d₆) δ 8.47-8.45 (1H, m), 8.30 (1H, d, J=8.4 Hz), 7.98-7.93 (2H, m), 7.32-6.83 (10H, m), 3.82 (3H, s), 2.72 (2H, s).

### Comparative Example 1

### 3-phenyl-1-(pyridin-2-yl)-1H-pyrazol-5-ol (89403)

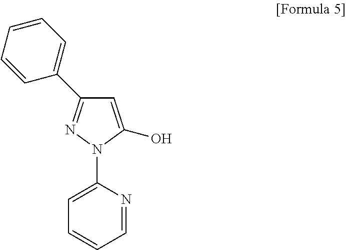 [SMILES: Oc1cc(-c2ccccc2)nn1-c1ccccn1]

3-phenyl-1-(pyridin-2-yl)-1H-pyrazol-5-ol was synthesized by the method disclosed in Korean Patent No. KR 0942382.

Ethyl benzoylacetate (1 equivalent) and ethanol (4 mL) were placed in a round-bottom flask, and 2-hydrazinopyridine (1.1 equivalents) diluted with ethanol (3 mL) was slowly added dropwise at 0° C. The resulting solution was heated under reflux for 20 minutes. The solvent was removed by distillation under reduced pressure, and the resulting solid was washed with hexane and ethyl acetate and dried under vacuum to give the target compound in 87% yield.

### Experimental Example 1

### Determination of Inhibitory Effect of Compounds on Migration of Smooth Muscle Cells

In order to determine the effect of the compounds of the present invention on the migration of smooth muscle cells (SMCs), human aortic smooth muscle cells were stimulated with LPS, and the migration of the cells was measured using a Transwell system. Here, experimental groups, in which smooth muscle cells were treated with the compounds synthesized in the above Examples ((278) in Example 1, (305) in Example 2, (306) in Example 3, and (308) in Example 4) in a concentration of 10 μM, and control groups, including a group in which smooth muscle cells were treated with the compound (89403) of Comparative Example 1 in the same concentration as the compounds of the Examples, a group ((−)LPS) which was not treated with the compounds synthesized in the Examples or Comparative Example and not stimulated with LPS, and a group ((+)LPS) which was not treated with the compounds synthesized in the Examples or Comparative Example and stimulated with LPS only, were compared.

Specifically, the underside of the upper layer of a Transwell (Costar Corning, Cambridge, Mass.) was coated with fibronectin, and then human aortic smooth muscle cells were plated in the upper chamber of the Transwell (8×10³). The upper chamber was pre-incubated on a plate containing the following compound for 30 minutes and then incubated for 16 hours.

| | | Added Materials (amount) |
|---|---|---|
| Control | (−) LPS | DMSO (10 uM) |
| groups | (+) LPS | DMSO (10 uM) + LPS (100 μg/ml) |
| | 89403 | LPS (100 μg/ml) + Compound in Comparative |
| | | Example (10 μM) |
| Experimental | 278 | LPS (100 μg/ml) + Compound synthesized in |
| groups | | Example 1 (10 μM) |
| | 305 | LPS (100 μg/ml) + Compound synthesized in |
| | | Example 2 (10 μM) |
| | 306 | LPS (100 μg/ml) + Compound synthesized in |
| | | Example 3 (10 μM) |
| | 308 | LPS (100 μg/ml) + Compound synthesized in |
| | | Example 3 (10 μM) |

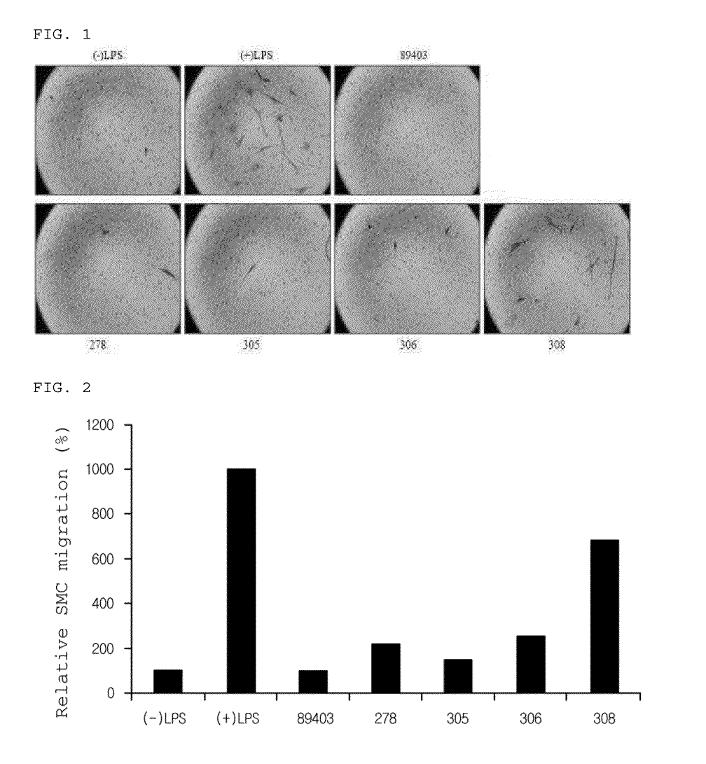

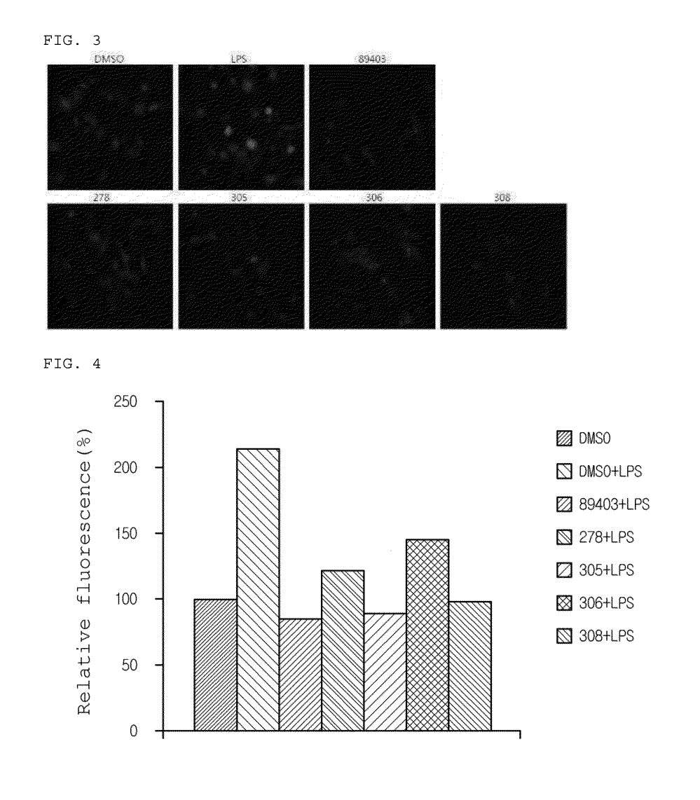

Then, the human aortic smooth muscle cells migrating through the Transwell were fixed in 70% methanol for 1 minute, stained with hematoxylin for 5 minutes, and then stained with eosin for 2 minutes. The human aortic smooth muscle cells that did not migrate and remained were removed, and then the migration degree of only the migrated human aortic smooth muscle cells were observed under a microscope (FIG. 1) and counted to compare the degree of cell migration (FIG. 2).

As can be seen from FIGS. 1 and 2, the experimental groups effectively inhibited the migration of vascular aortic smooth muscle cells, compared to the group treated with LPS alone ((+) LPS). Therefore, it can be seen that the composition comprising a pyrazole derivative of the present invention has a prevention effect on vascular restenosis and a therapeutic effect on cardiovascular diseases such as arteriosclerosis by effectively inhibiting the migration of vascular aortic smooth muscle cells.

### Experimental Example 2

### Determination of Inhibitory Effect of Compounds on Generation of Reactive Oxygen Species (ROS)— under Confocal Microscope

Human aortic smooth muscle cells (2×10⁵) were plated in a 35 mm dish and incubated for one day. The resulting cells were subjected to serum starvation for 16 hours and classified into samples of seven groups as follows. Each sample of groups was pre-incubated with each compound for 30 minutes and then treated with LPS for 20 minutes.

| | | | LPS (100 μg/ml) |
|---|---|---|---|
| | | Pre-incubation (30 min) | treatment (20 min) |
| Control | DMSO | DMSO (10 μm) | Non-treated |
| groups | LPS | DMSO (10 μm) | Treated |
| | 89403 | Compound in Comparative | Treated |
| | | Example (10 μM) | |
| Experimental | 278 | Compound synthesized in | Treated |
| groups | | Example 1 (10 μM) | |
| | 305 | Compound synthesized in | Treated |
| | | Example 2 (10 μM) | |
| | 306 | Compound synthesized in | Treated |
| | | Example 3 (10 μM) | |
| | 308 | Compound synthesized in | Treated |
| | | Example 4 (10 μM) | |

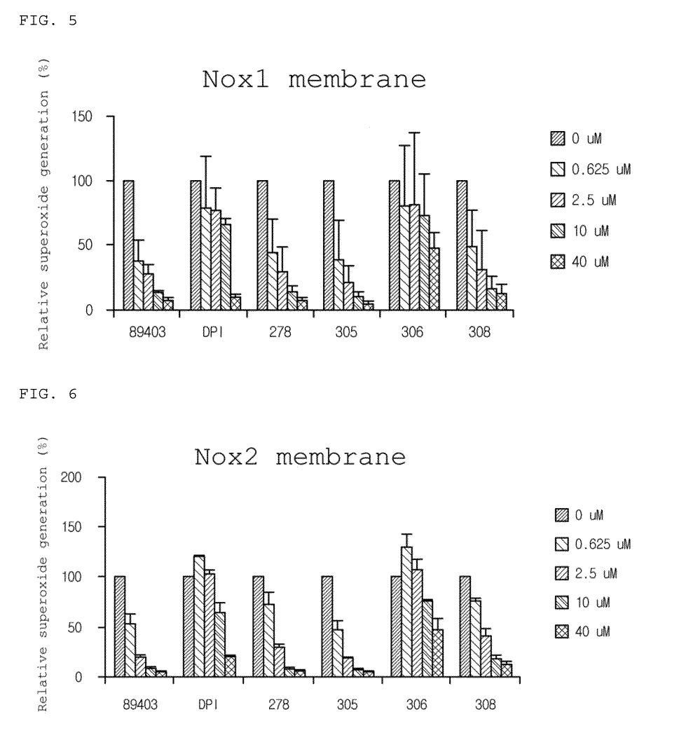

Then, the resulting sample cells were stained with 10 μM 2′,7′-dichlorodihydrofluorescein diacetate (DCF-DA) in dark for 10 minutes, and their images were observed with an argon laser (excitation wavelength: 488 nm, emission filter: 515 to 540 nm) equipped with a confocal microscope (LSM510, Version 2.3) (FIG. 3).

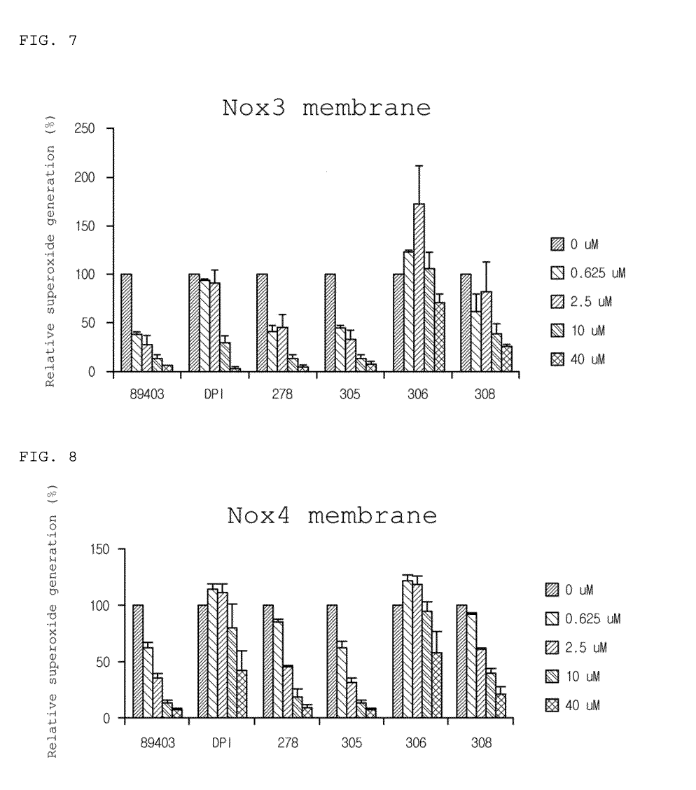

Five images were obtained from each sample, and the fluorescence intensity was measured with a Zeiss vision system (LSM510, Version 2.3). Relative fluorescence intensity values were calculated based on the DMSO control by substituting the obtained intensity values into the following formula and the results are shown in FIG. 4.

Human aortic smooth muscle cells (2×10⁵) were plated in a 35 mm dish and incubated for one day. The resulting cells were subjected to starvation in serum-free media for 16 hours, stimulated with 100 pg/ml LPS for 20 minutes, and stained with 10 μM 2′,7′-dichlorodihydrofluorescein diacetate (DCF-DA) in dark for 10 minutes, and their images were observed with an argon laser (excitation wavelength: 488 nm, emission filter: 515 to 540 nm) equipped with a confocal microscope (LSM510, Version 2.3). Five images were obtained from each sample, and the fluorescence intensity was measured with a Zeiss vision system (LSM510, Version 2.3). The obtained intensity values were converted into relative fluorescence intensity values based on the DMSO control and plotted.

$$Relative fluorescence = \frac{LPS or each chemical + LPS sample fluorescence intensity}{DMSO sample fluorescence intensity} \times 100$$

As can be seen from FIGS. 3 and 4, relative fluorescence intensity decreased in the experimental groups, compared to that in the control group treated with LPS alone, and the experimental groups showed the relative fluorescence intensity similar to that of the control group with a proven therapeutic effect on atherosclerosis (Korean Patent No. 10-0987557). Therefore, it can be seen that the composition comprising a pyrazole derivative of the present invention has a preventive effect on vascular restenosis and a therapeutic effect on cardiovascular diseases such as arteriosclerosis by effectively inhibiting the generation of reactive oxygen species (ROS).

### Experimental Example 3

### Determination of Inhibitory Effect of Compounds on Generation of Reactive Oxygen Species (ROS)— Lucigenin-based Assay

(1) Isolation of Plasma Membranes from Drosophila Embryos

Plasma membranes were isolated from transgenic drosophila over-expressing human Nox1/Nox2/Nox3/Nox4/Duox1/Duox2 as follows.

Drosophila was placed in PBS buffer, and its tissue was disrupted with a homogenizer, sonicated, and centrifuged at 10000 g for 3 minutes. The supernatant was collected and centrifuged at 4° C. and 100000 g for 1 hour to obtain a pellet, and the pellet was resuspended in PBS to obtain Nox1/Nox2/Nox3/Nox4/Duox1/Duox2 Nox enzymes, respectively.

(2) Measurement of the Amount of Superoxide Generated

The Nox1 membrane obtained in (1) of Experimental Example 3 was treated with the compounds synthesized in the above Examples ((278) in Example 1, (305) in Example 2, (306) in Example 3, and (308) in Example 4) at each concentration in experimental groups and treated with the compound (89403) of Comparative Example 1 and DMSO control at each concentration in control groups. At the same time, 500 μM NADH and 500 μM lucigenin were added to each group, and the amount of superoxide generated at 28° C. for 15 minutes was measured using a Multimode detector (DTX 880, Beckman coulter).

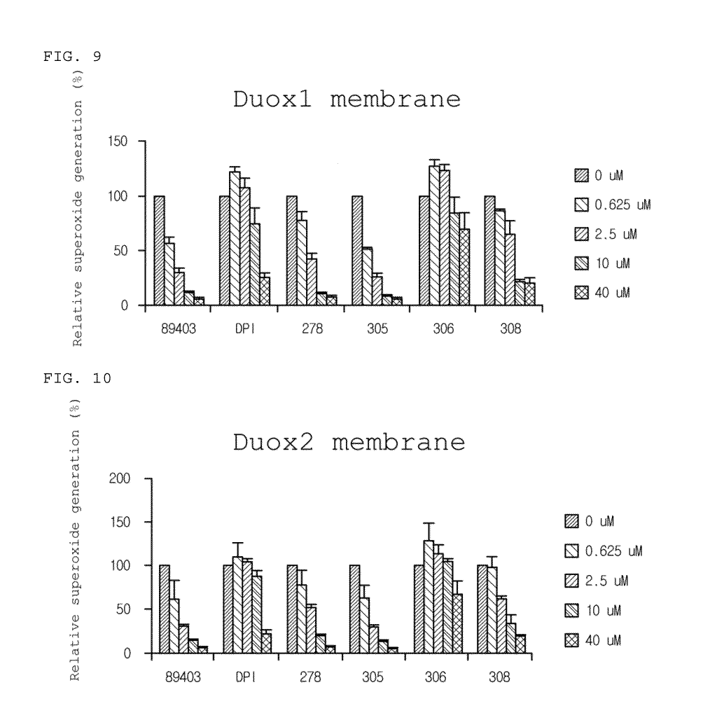

The amount of superoxide generated was substituted into the following formula to calculate the relative superoxide generation, and the results are shown in FIG. 5.

$$Relative superoxide generation ( \% ) = \frac{The amount of superoxide generation of each sample}{The amount of superoxide generation of DMSO control} \times 100$$

Moreover, the amount of superoxide was measured from the Nox2/Nox3/Nox4/Duox1/Duox2 membranes obtained in (1) of Experimental Example 3, respectively, in the same manner. The results are shown in FIGS. 6 to 10.

As can be seen from FIGS. 5 to 10, all experimental groups showed the relative superoxide generation with respect to all Nox1/Nox2/Nox3/Nox4/Duox1/Duox2 membranes similar to that of the control group with a proven therapeutic effect on atherosclerosis (Korean Patent No. 10-0987557).

Therefore, it can be seen that the composition comprising a pyrazole derivative of the present invention has a preventive effect on vascular restenosis and a therapeutic effect on cardiovascular diseases such as arteriosclerosis by effectively inhibiting the generation of reactive oxygen species (ROS).

### INDUSTRIAL APPLICABILITY

The compounds of the present invention have excellent inhibitory activity on the generation of reactive oxygen species and can be used for the treatment or prevention of cardiovascular diseases without any special side effects of conventional therapeutic agents.

1. A composition for preventing or treating cardiovascular diseases, comprising a compound represented by the following formula 1: 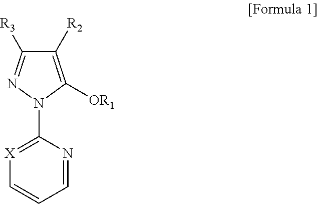
wherein X represents —CH— or nitrogen;
R₁ represents a hydrogen atom or an isopropyloxycarbonyloxymethyl;
R₂ represents a hydrogen atom, a C1-C4 linear or branched alkyl, or a substituted or unsubstituted benzyl; and
R₃ represents a phenyl, a nitrophenyl, a substituted or unsubstituted phenylethenyl, or a substituted or unsubstituted diphenylethenyl;
wherein when X represents —CH— and R₁ and R₂ each represents a hydrogen atom, R₃ represents a substituted or unsubstituted diphenylethenyl, or when X represents a nitrogen atom and R₁ and R₂ each represents a hydrogen atom, R₃ represents a nitrophenyl or a substituted or unsubstituted diphenylethenyl group; and
wherein the substituent is a nitro, a hydroxyl, or a methoxyl.

2. A composition for preventing or treating cardiovascular diseases, comprising a pharmaceutically acceptable salt of a compound represented by the following formula 1: 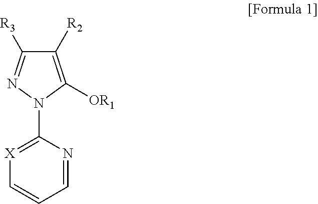
wherein X represents —CH— or nitrogen;
R₁ represents a hydrogen atom or an isopropyloxycarbonyloxymethyl;
R₂ represents a hydrogen atom, a C1-C4 linear or branched alkyl, or a substituted or unsubstituted benzyl; and
R₃ represents a phenyl, a nitrophenyl, a substituted or unsubstituted phenylethenyl, or a substituted or unsubstituted diphenylethenyl;
wherein when R₁ and R₂ each represents a hydrogen atom, R₃ represents a phenyl group, a nitrophenyl group, or a substituted or unsubstituted diphenylethenyl; and
wherein the substituent is a nitro, a hydroxyl, or a methoxyl.

3. A pharmaceutical composition for preventing or treating cardiovascular diseases, containing a compound represented by the following formula I or a pharmaceutically acceptable salt thereof: 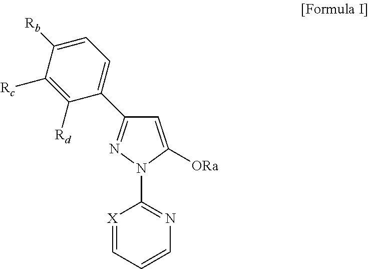
wherein X represents —CH— or nitrogen;
Rₐ represents a hydrogen atom, an acetyl group, a tri(C1-C4)alkylsilanyl group, a diphenylboranyl group, or a (t-butoxy)carbonyl group; and
R_{b}, R_{c} and R_{d} each independently represents a hydrogen atom, a halogen atom, a halo(C1-C3)alkyl group, a (C1-C6)alkoxy group, a benzo[d][1,3]dioxol group, a substituted or unsubstituted biphenyl group, or a substituted or unsubstituted (C6-C10)aryl group; and
wherein the substituent is a halogen atom, a (C1-C4)alkyl amine group, a halo(C1-C3)alkyl group, a (C1-C6)alkoxy group, a phenoxy group, a benzyloxy group, a formyl group, or a halogen-substituted phenyl group, with the proviso that R_{b}, R_{c} and R_{d} are not a hydrogen atom at the same time.

4. A pharmaceutical composition for preventing or treating cardiovascular diseases, comprising a compound represented by the following formula A or a pharmaceutically acceptable salt thereof: 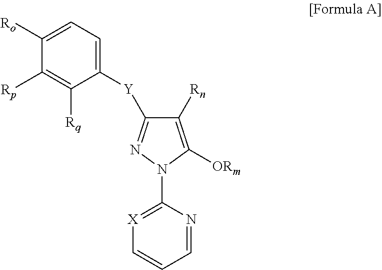
wherein X represents —CH— or nitrogen;
Y represents —CH₂—, —CH₂CH₂—, —CH═CH—, —CH₂—O—, or —O—CH₂—;
Rₘ represents a hydrogen atom, an acetyl group, a tri(C1-C4)alkylsilanyl group, a diarylboranyl group, or a (t-butoxy)carbamyl group;
Rₙ represents a hydrogen atom or a (C1-C4)alkyl group; and
Rₒ, Rₚ and R_{q} each independently represents a hydrogen atom, a hydroxyl group, a halogen atom, a (C6-C10)aryl group, a halo(C1-C3)alkyl group, a (C1-C6)alkoxy group, a tri(C1-C4)alkylsilanoxy group, or a benzodioxolyl group; or
alternatively Rₒ and Rₚ or Rₚ and R_{q} together represent —CH₂—CH═CH—, —CH═CH—CH═CH—, or —CH═CH—CH═CH—CH₂—.

5. A pharmaceutical composition for preventing or treating cardiovascular diseases, comprising at least one compound selected from the group consisting of:
1-(pyridin-2-yl)-3-phenyl-4-propyl-M-pyrazol-5-ol (211),
1-(pyridin-2-yl)-3-phenyl-4-benzyl-1H-pyrazol-5-ol (212),
1-(pyridin-2-yl)-3-phenyl-4-(4-nitrobenzyl)/H-pyrazol-5-ol (225),
1-(pyridin-2-yl)-3-phenyl-4-propyl-1H-5-(isopropyloxycarbonyloxymethyloxy)pyrazole (306),
1-(pyridin-2-yl)-3-[(3-methoxy-4-hydroxyphenyl)-E-ethenyl]-4-benzyl-1H-pyrazol-5-ol,
1-(pyridin-2-yl)-3-[(3-methoxy-4-hydroxyphenyl)-E-ethenyl]-4-benzyl-5-hydroxypyrazole (274),
3-(4-nitrophenyl)-3-[2-(pyrimidin-2-yl)hydrazono]propionic acid ethyl ester,
1-(pyrimidin-2-yl)-3-(4-nitrophenyl)-5-1H-pyrazol-5-ol (105),
1-(pyridin-2-yl)-3-(1,2-diphenyl-E-ethenyl)-1H-pyrazol-5-ol (263),
3-(4-methoxyphenylamino)-1H-pyrazol-5-ol (15),
4-hexylidene-3-phenyl-1-(pyridin-2-yl)-1H-pyrazol-5-ol (213),
1-(pyridin-2-yl)-3-(3-(trifluoromethyl)phenyl)-1H-pyrazol-5-ol,
1-(pyridin-2-yl)-3-(4-(trifluoromethyl)phenyl)-1H-pyrazol-5-ol,
3-(3-nitrophenyl)-1-(pyridin-2-yl)-1H-pyrazol-5-ol,
3-(4-nitrophenyl)-1-(pyridin-2-yl)-1H-pyrazol-5-ol,
3-(4-methoxyphenyl)-1-(pyridin-2-yl)-1H-pyrazol-5-ol,
3-(3,4-dimethoxyphenyl)-1-(pyridin-2-yl)-1H-pyrazol-5-ol,
1-(pyridin-2-yl)-3-(3,4,5-trimethoxyphenyl)-1H-pyrazol-5-ol,
3-(2-fluorophenyl)-1-(pyridin-2-yl)-1H-pyrazol-5-ol,
3-(3-fluorophenyl)-1-(pyridin-2-yl)-1H-pyrazol-5-ol,
3-(4-fluorophenyl)-1-(pyridin-2-yl)-1H-pyrazol-5-ol,
3-(2-chlorophenyl)-1-(pyridin-2-yl)-1H-pyrazol-5-ol,
3-(3-chlorophenyl)-1-(pyridin-2-yl)-1H-pyrazol-5-ol,
3-(4-chlorophenyl)-1-(pyridin-2-yl)-1H-pyrazol-5-ol,
3-(3-bromophenyl)-1-(pyridin-2-yl)-1H-pyrazol-5-ol,
3-(4-bromophenyl)-1-(pyridin-2-yl)-1H-pyrazol-5-ol,
3-(2-iodophenyl)-1-(pyridin-2-yl)-1H-pyrazol-5-ol,
3-(3-iodophenyl)-1-(pyridin-2-yl)-1H-pyrazol-5-ol,
3-(4-iodophenyl)-1-(pyridin-2-yl)-1H-pyrazol-5-ol,
3-(4-iodophenyl)-1-(pyrimidin-2-yl)-1H-pyrazol-5-ol,
2-(3-(4-iodophenyl)-5-(triisopropylsilyloxy)-1H-pyrazol-1-yl)pyridine,
3-(4-bromophenyl)-1-(pyridin-2-yl)-1H-pyrazol-5-yl t-butyl carbonate,
t-butyl 3-(2-iodophenyl)-1-(pyridin-2-yl)-1H-pyrazol-5-yl carbonate,
t-butyl 3-(3-iodophenyl)-1-(pyridin-2-yl)-1H-pyrazol-5-yl carbonate,
t-butyl 3-(4-iodophenyl)-1-(pyridin-2-yl)-1H-pyrazol-5-yl carbonate,
t-butyl 3-(4-iodophenyl)-1-(pyrimidin-2-yl)-1H-pyrazol-5-yl carbonate,
3-(4-bromophenyl)-1-(pyridin-2-yl)-1H-pyrazol-5-yl acetate,
3-(biphenyl-4-yl)-1-(pyridin-2-yl)-1H-pyrazol-5-yl t-butyl carbonate,
3-(4-(benzo[d][1,3]dioxol-5-yl)phenyl)-1-(pyridin-2-yl)-1H-pyrazol-5-yl t-butyl carbonate,
3-(3′-phenylbiphenyl-4-yl)-1-(pyridin-2-yl)-1H-pyrazol-5-yl t-butyl carbonate,
t-butyl 3-(4-(naphthalene)phenyl)-1-(pyridin-2-yl)-1H-pyrazol-5-yl carbonate,
t-butyl 3-(3′-(dimethylamino)biphenyl)-1-(pyridin-2-yl)-1H-pyrazol-5-yl carbonate,
3-(2-(benzo[d][1,3]dioxol-5-yl)phenyl)-1-(pyridin-2-yl)-1H-pyrazol-5-yl t-butyl carbonate,
3-(3′-phenylbiphenyl-2-yl)-1-(pyridin-2-yl)-1H-pyrazol-5-yl t-butyl carbonate,
3-(biphenyl-3-yl)-1-(pyridin-2-yl)-1H-pyrazol-5-yl t-butyl carbonate,
3-(4′-(benzyloxy)biphenyl-3-yl)-1-(pyridin-2-yl)-1H-pyrazol-5-yl t-butyl carbonate,
3-(4′-bromobiphenyl-3-yl)-1-(pyridin-2-yl)-1H-pyrazol-5-yl t-butyl carbonate,
t-butyl 3-(3′-formylbiphenyl-3-yl)-1-(pyridin-2-yl)-1H-pyrazol-5-yl carbonate,
t-butyl 3-(2′-phenoxybiphenyl-3-yl)-1-(pyridin-2-yl)-1H-pyrazol-5-yl carbonate,
3-(3-(benzo[d][1,3]dioxol-5-yl)phenyl)-1-(pyridin-2-yl)-1H-pyrazol-5-yl t-butyl carbonate,
3-(3′-phenylbiphenyl-3-yl)-1-(pyridin-2-yl)-1H-pyrazol-5-yl t-butyl carbonate,
t-butyl 3-(3-(naphthalene-1-yl)phenyl)-1-(pyridin-2-yl)-1H-pyrazol-5-yl carbonate,
t-butyl 3-(3′-(dimethylamino)biphenyl-3-yl)-1-(pyridin-2-yl)-1H-pyrazol-5-yl carbonate,
t-butyl 3-(4′-methoxybiphenyl-4-yl)-1-(pyridin-2-yl)-1H-pyrazol-5-yl carbonate,
3-(4′-(benzyloxy)biphenyl-4-yl)-1-(pyridin-2-yl)-1H-pyrazol-5-yl t-butyl carbonate,
3-(4′-bromobiphenyl-4-yl)-1-(pyridin-2-yl)-1H-pyrazol-5-yl t-butyl carbonate,
3-(3′-formylbiphenyl-4-yl)-1-(pyridin-2-yl)-1H-pyrazol-5-yl t-butyl carbonate,
t-butyl 3-(2′-phenoxybiphenyl-4-yl)-1-(pyridin-2-yl)-1H-pyrazol-5-yl carbonate,
3-(biphenyl-4-yl)-1-(pyrimidin-2-yl)-1H-pyrazol-5-yl t-butyl carbonate,
3-(4-(benzo[d][1,3]dioxol-5-yl)phenyl)-1-(pyrimidin-2-yl)-1H-pyrazol-5-yl t-butyl carbonate,
3-(3′-phenylbiphenyl-4-yl)-1-(pyrimidin-2-yl)-1H-pyrazol-5-yl t-butyl carbonate,
2-(3-(4′-bromobiphenyl-4-yl)-5-(triisopropylsilyloxy)-1H-pyrazol-1-yl)pyridine,
2-(3-(biphenyl-4-yl)-5-(diphenylboryloxy)-1H-pyrazol-1-yl)pyridine,
3-(biphenyl-4-yl)-1-(pyridin-2-yl)-1H-pyrazol-5-ol,
3-(4-(benzo[d][1,3]dioxol-5-yl)phenyl)-1-(pyridin-2-yl)-1H-pyrazol-5-ol,
3-(3′-phenylbiphenyl-4-yl)-1-(pyridin-2-yl)-1H-pyrazol-5-ol,
3-(4-(naphthalene-1-yl)phenyl)-1-(pyridin-2-yl)-1H-pyrazol-5-ol,
3-(3′-(dimethylamino)biphenyl-4-yl)-1-(pyridin-2-yl)-1H-pyrazol-5-ol,
3-(2-(benzo[d][1,3]dioxol-5-yl)phenyl)-1-(pyridin-2-yl)-1H-pyrazol-5-ol,
3-(3′-phenylbiphenyl-2-yl)-1-(pyridin-2-yl)-1H-pyrazol-5-ol,
3-(biphenyl-3-yl)-1-(pyridin-2-yl)-1H-pyrazol-5-ol,
3-(4′-(benzyloxy)biphenyl-3-yl)-1-(pyridin-2-yl)-1H-pyrazol-5-ol,
3-(4′-bromobiphenyl-3-yl)-1-(pyridin-2-yl)-1H-pyrazol-5-ol,
3′-(3-formylbiphenyl-3-yl)-1-(pyridin-2-yl)-1H-pyrazol-5-ol,
3-(2′-phenoxybiphenyl-3-yl)-1-(pyridin-2-yl)-1H-pyrazol-5-ol,
3-(3-(benzo[d][1,3]dioxol-5-yl)phenyl)-1-(pyridin-2-yl)-1H-pyrazol-5-ol,
3-(3′-phenylbiphenyl-3-yl)-1-(pyridin-2-yl)-1H-pyrazol-5-ol,
3-(3-(naphthalene-1-yl)phenyl)-1-(pyridin-2-yl)-1H-pyrazol-5-ol,
3-(3′-(dimethylamino)biphenyl-3-yl)-1-(pyridin-2-yl)-1H-pyrazol-5-ol,
3-(4′-methoxybiphenyl-4-yl)-1-(pyridin-2-yl)-1H-pyrazol-5-ol,
3-(4′-(benzyloxy)biphenyl-4-yl)-1-(pyridin-2-yl)-1H-pyrazol-5-ol,
3-(4′-bromobiphenyl-4-yl)-1-(pyridin-2-yl)-1H-pyrazol-5-ol,
3-(3′-formylbiphenyl-4-yl)-1-(pyridin-2-yl)-1H-pyrazol-5-ol,
3-(2′-phenoxybiphenyl-4-yl)-1-(pyridin-2-yl)-1H-pyrazol-5-ol,
3-(biphenyl-4-yl)-1-(pyrimidin-2-yl)-1H-pyrazol-5-ol,
3-(4-(benzo[d][1,3]dioxol-5-yl)phenyl)-1-(pyrimidin-2-yl)-1H-pyrazol-5-ol,
3-(3′-phenylbiphenyl-4-yl)-1-(pyridin-2-yl)-1H-pyrazol-5-ol,
3-phenyl-4-propyl-1-(pyridin-2-yl)-1H-pyrazol-5-ol,
4-benzyl-3-phenyl-1-(pyridin-2-yl)-1H-pyrazol-5-ol,
3-benzyl-1-(pyridin-2-yl)-1H-pyrazol-5-ol,
3-(o-bromobenzyl)-1-(pyridin-2-yl)-1H-pyrazol-5-ol,
3-(p-methoxybenzyl)-1-(pyridin-2-yl)-1H-pyrazol-5-ol,
3-phenethyl-1-(pyridin-2-yl)-1H-pyrazol-5-ol,
3-(p-chlorobenzyl)-1-(pyridin-2-yl)-1H-pyrazol-5-ol,
3-(phenoxymethyl)-1-(pyridin-2-yl)-1H-pyrazol-5-ol,
3-(naphthalene-3-yloxy)methyl)-1-(pyridin-2-yl)-1H-pyrazol-5-ol,
3-((4-chlorophenoxy)methyl)-1-(pyridin-2-yl)-1H-pyrazol-5-ol,
3-((2,4-dichlorophenoxymethyl)-1-(pyridin-2-yl)-1H-pyrazol-5-ol,
1-(pyridin-2-yl)-3-styryl-1H-pyrazol-5-ol,
3-(4-methoxystyryl)-1-(pyridin-2-yl)-1H-pyrazol-5-ol,
3-(3,4-dimethoxystyryl)-1-(pyridin-2-yl)-1H-pyrazol-5-ol,
3-(3,4-dichlorostyryl)-1-(pyridin-2-yl)-1H-pyrazol-5-ol,
3-(4-i-propylstyryl)-1-(pyridin-2-yl)-1H-pyrazol-5-ol,
3-(4-trifluoromethylstyryl)-1-(pyridin-2-yl)-1H-pyrazol-5-ol,
3-(4-t-butyldimethylsilyloxystyryl)-1-(pyridin-2-yl)-1H-pyrazol-5-ol,
3-(3-methoxy-4-t-butyldimethylsilyloxystyryl)-1-(pyridin-2-yl)-1H-pyrazol-5-ol,
3-(3,5-dimethoxy-4-t-butyldimethylsilyloxystyryl)-1-(pyridin-2-yl)-1H-pyrazol-5-ol,
3-(3-methoxy-4-t-butyldimethylsilyloxystyryl)-1-(pyridin-2-yl)-1H-pyrazol-5-thiol,
3-(4-hydroxystyryl)-1-(pyridin-2-yl)-1H-pyrazol-5-ol,
3-(3-methoxy-4-hydroxystyryl)-1-(pyridin-2-yl)-1H-pyrazol-5-ol,
3-(3,5-dimethoxy-4-hydroxystyryl)-1-(pyridin-2-yl)-1H-pyrazol-5-ol,
3-(3-methoxy-4-hydroxystyryl)-1-(pyridin-2-yl)-1H-pyrazol-5-thiol, and
4-((E)-2-(5-amino-1-(pyridin-2-yl)-1H-pyrazol-3-yl)vinyl)-2-methoxyphenol;
or a pharmaceutically acceptable salt thereof.

6. The pharmaceutical composition of claim 5, wherein the pharmaceutical composition comprises at least one compound selected from the group consisting of:
1-(pyridin-2-yl)-3-phenyl-4-propyl-1H-pyrazol-5-ol,
1-(pyridin-2-yl)-3-phenyl-4-propyl-1H-5-(isopropyloxycarbonyloxymethyloxy)pyrazole (306), and
1-(pyridin-2-yl)-3-[(3-methoxy-4-hydroxyphenyl)-E-ethenyl]-4-benzyl-1-pyrazol 5-ol;
or a pharmaceutical) acceptable salt thereof.

7. A method for removing vascular stenosis, the method comprising administering to a subject in need thereof a composition of any one of claims 1 to 6.

8. A drug delivery system for topical administration of a composition for preventing or treating vascular restenosis, comprising at least one selected from: a compound of formula 1 or a pharmaceutically acceptable salt thereof; a compound of formula I or a pharmaceutically acceptable salt thereof; and a compound of formula A or a pharmaceutically acceptable salt thereof.

9. A method for removing vascular stenosis, the method comprising administering to a subject in need thereof at least one selected from the group consisting of:
1-(pyridin-2-yl)-3-phenyl-4-propyl-M-pyrazol-5-ol hydrochloride,
1-(pyridin-2-yl)-3-phenyl-M-pyrazol-5-ol hydrochloride, and
1-(pyridin-2-yl)-3-[(3-methoxy-4-hydroxyphenyl)-E-ethenyl]-4-benzyl-M-pyrazol-5-ol hydrochloride.
